(19) United States
(12) Patent Application Publication
(10) Pub. No.: US 2012/0101099 A1
(43) Pub. Date: Apr. 26, 2012

(54) HISTONE DEACETYLASE INHIBITORS

(75) Inventors: Natesan Selvakumar, Bangalore (IN); Gavara Govinda Rajulu, Bangalore (IN); Annamalai Pazhanimuthu, Hosur (IN); Sambasivam Ganesh, Bangalore (IN)

(73) Assignee: ANTHEM BIOSCIENCES PRIVATE LIMITED, Bangalore, Karnataka (IN)

(21) Appl. No.: 13/382,584

(22) PCT Filed: Jul. 7, 2010
(86) PCT No.: PCT/IN2010/000455

§ 371 Date: Jan. 6, 2012

(30) Foreign Application Priority Data
Jul. 7, 2009 (IN) 1610/CHE/2009

(51) Int. Cl. A61K 31/4192; C07D 401/04; C12N 9/99; C07D 413/10; A61K 31/5377; A61P 35/00; C07D 249/06; A61K 31/4439

(52) U.S. Cl. 514/236.2; 548/255; 514/359; 546/268.4; 514/340; 544/132; 435/184

(57) ABSTRACT

The disclosure provides compounds of formula I and methods for preparation thereof. The compounds act as inhibitor of histone deacetylase.

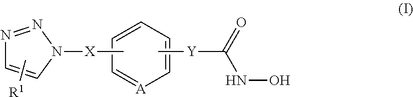

### TECHNICAL FIELD

The present disclosure relates to hydroxamate compounds that are inhibitors of histone deacetylase having general formula (I). More particularly, the present disclosure relates to triazole comprising compounds and methods for their preparation. These compounds may be useful as medicaments for the treatment of proliferative disorders as well as other diseases involving, relating to or associated with dysregulation of histone deacetylase (HDAC).

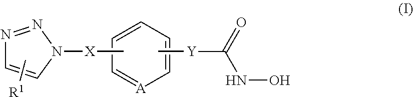

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

X is either absent or is selected from a group comprising cycloalkyl, —(CH₂)ₙ—, —(CH)ₙR^{a}—, —(CH₂)ₙ—NR^{b}—CO—(CH₂)ₙ—, —(CH)ₙR^{a}—NR^{b}—CO—(CH₂)ₙ—, —(CH)ₙR^{a}—NR^{b}—CO—(CH)ₙR^{c}—, —(CH₂)ₙ—NR^{b}—CO—(CH)ₙR^{c}—, —(CH₂)ₙ—NR^{b}—CO—(CH₂)ₙ—, —(CH)ₙR^{a}—NR^{b}—SO₂—(CH₂)ₙ—, —(CH)ₙR^{a}—NR^{b}—SO₂—(CH)ₙR^{c}— and —(CH₂)ₙ—NR^{b}—SO₂—(CH)ₙR^{c}—;

n is an integer selected from 0 to 6;

R^{a} and R^{c} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl;

R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, —C(═O)R^{a}, —C(═O)OR^{a}, —C(═O)NR^{a}R^{c} and —SO₂R^{a};

Y is either absent or selected from a group comprising —CH₂—, —CH₂CH₂—, —CH═CH—, C₃-C₆ cycloalkyl each of which is optionally substituted with a substituent selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl; and -A is selected from a group comprising Carbon and Nitrogen.

The present disclosure also provides a process for the preparation of the above said compounds of the general formula (I).

### BACKGROUND

The present disclosure relates to potential compounds of formula (I), pharmaceutical compositions that can be used in particular as anticancer agents. Compounds of the general formula (I), or pharmaceutically acceptable salts thereof according to the present disclosure have an ability of inhibiting histone deacetylating enzyme and of inducing differentiation and are useful as therapeutic or ameliorating agent for diseases that are involved in cellular growth such as malignant tumors, autoimmune diseases, skin diseases, infections.

The field of histone deacetylase inhibitors is moving into a new phase of development. The exponential growth in the level of research activity surrounding the histone deacetylases (HDACs) witnessed over the past decade has now started to produce success in the clinic, particularly in the field of oncology

The HDAC inhibitors have been driven by their ability to modulate transcriptional activity. As a result, this therapeutic class is able to block angiogenesis and cell cycling, and promote apoptosis and differentiation. By targeting these key components of tumor proliferation, HDAC inhibitors have the potential to occupy an indomitable position in the fast-moving anticancer market.

Although HDAC inhibitors display targeted anticancer activity per se, a major reason why this class could play such a key role in oncology is that HDAC inhibition is able to improve the efficacy of existing agents as well as other new targeted therapies. Over the past few years, a handful of HDAC inhibitors have entered the clinic and the overall opinion is that these candidates are relatively safe.

Cancer is the second major disease causing deaths all over the world. American cancer society estimated 0.59 million deaths in 2007. The major types are accounted to 30% of Lung cancer, 15% of breast cancer, 10% of colon and rectal, 9% of prostate cancer and 6% each to pancreas, ovary and leukemia. It is estimated that there will be 16 million new cases every year by 2020. Cancer causes 7 million deaths every year or 12.5% of deaths worldwide.

Histone acetylation/deacetylation is essential for chromatin remodeling, regulation of gene transcription and gene expression. HDACs (EC number 3.5.1) are a class of enzymes that remove acetyl groups from the c-N-acetyl lysine amino acid.

HDACs are grouped into class I, class II, class III and class IV based on their sequence homology to their yeast orthologues Rpd3, HdaI and Sir2 [A. J. de Ruijter, Biochem. J. 370, 737-749 (2003)].

Basic biochemical functions of HDAC are deaceylating the lysine residues of the histone proteins and numerous non histone substrate proteins which play a critical role in gene regulation, cell cycle, angiogenesis, differentiation and apoptosis [Adam G. Inche Drug Discov Today. 11, 97-109 (2006)].

In general, increased levels of histone acetylation are associated with increased transcriptional activity, whereas decreased levels of acetylation are associated with repression of gene expression [Wade P. A. Hum. Mol. Genet. 10, 693-698 (2001)]

Aberrant activity and over expression of HDACs were reported in several cancer cell lines. [J H Choi J H Jpn J Cancer Res 92, 1300-4 (2001) and Samir K. Patra Biochem Biophys Res Commun. 287, 705-13 (2001)]

HDAC inhibitors serve as target based non cytotoxic agents which can bring both safety and efficacy to the patients over the other anticancer drugs. [M A Glozak, Oncogene 26, 5420-5432 (2007)]

HDAC inhibitors are promising agents for cancer therapy as effective inducers of apoptosis. Several structural classes of HDAC inhibitors (HDACIs) have been identified and are reviewed in Marks, P. A. et al., J. Natl. Cancer Inst., 92, (2000), 1210-1215. More specifically the patents WO 98/55449 and U.S. Pat. No. 5,369,108 report alkanoyl hydroxamates with HDAC inhibitory activity. HDACIs currently in clinical development cover pan-HDACIs (Vorinostat, Belinostat, and LBH589) and somewhat isotype selective agents (Romidepsin, MS-275 and MGCDO 103) With the approval of Zolinza (Vorinostat, SAHA) by the FDA on October 2006 for the treatment of CTCL and with other histone deacetylase inhibitors awaiting approval for various cancers, this will hopefully prompt the investigation of histone deacetylase inhibitors into a broader range of disease states where altered chromatin function may play a role in their pathophysiology.

Over the next few years, experts believe that, the first generation HDAC inhibitors would produce clinical benefits while the second generation inhibitors could improve specificity. This class will emerge as a new class of cancer treatment. We have designed novel triazole based hydroxymic acids derivatives as potent HADC inhibitors and inhibiting the cancer cell proliferation. These finding suggest that inhibition of tumor cells HDACs represent the selective and novel non- cytotoxic therapy for the cancer
1) WO 02/22577 discloses following unsaturated hydroxamates as histone deacetylase inhibitors having general formula:

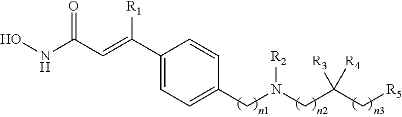

R₁ is H halo or a straight chain C₁-C₆ alkyl; R₂ is selected from H, C₁-C₁₀, C₄-C₉ cycloalkyl, C₄-C₉ heterocycloalkyl, C₄-C₉ heterocycloalkylalkyl, cycloalkylalkyl, aryl, heteroaryl, etc.; R₃ and R₄ are same or different and independently H, C₁-C₆ alkyl, acyl or acylamino, or R₃ and R₄ together with the carbon to which they are bound to represent C═O, C═S, etc., or R₂ together with the nitrogen to which it is bound and R₃ together with the carbon to which it is bound to form a C₄-C₉ heterocycloaryl, a heteroaryl, a polyheteroaryl, a non-aromatic polyheterocycle, or a mixed aryl and non-aryl polyheterocycle ring; R₅ is selected from H, C₁-C₆ alkyl, etc.; n, n₁, n₂ and n₃ are the same or different and independently selected from 0-6, each carbon can be optionally and independently substituted with R₃ and/or R₄; X and Y are the same or different and independently selected from H, halo, C₁-C₄ alkyl, etc.; or a pharmaceutically acceptable salt thereof.
2) WO2008076954 discloses histone deacetylase inhibitor compounds of formula:

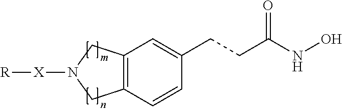

wherein the dashed line indicates a single or double bond, n and m are each, independently, 1, 2, or 3, and the sum of n and m is 2, 3 or 4; wherein X is (CH₂)ⱼ wherein each CH₂ may be independently replaced one or more times with C(O), S(O)₂, S(O), O, or NR₂, wherein R₂ is selected from the group consisting of H, alkyl, aryl, heterocycle, Cⱼ₋₄-alkyl, and C₃₋₆-cycloalkyl; j is an integer between 0 and 6.

R is selected from the group consisting of C₁₋₄-alkyl, C₃₋₆-cycloalkyl and aryl, wherein cycloalkyl and aryl may be further independently substituted one or more times with aryl, heterocycle, C₁₋₄-alkyl, halogen, amino, nitro, cyano, pyrrolidinyl or CF₃ (including pharmaceutically acceptable salts thereof).

### STATEMENT OF THE DISCLOSURE:

Accordingly the present disclosure provides a compound of formula (I),

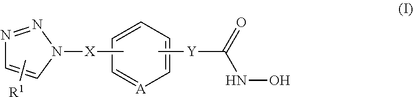

its derivatives, analogs, tautomeric forms, stereoisomers, polymorphs, solvates, salts, metabolites and prodrugs wherein, R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein, R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl, X is either absent or is selected from a group comprising cycloalkyl, —(CH₂)ₙ, —(CH)ₙR^{a}—, —(CH₂)ₙ—NR^{b}—CO—(CH₂)ₙ, —(CH)ₙR^{a}—NR^{b}—CO—(CH₂)ₙ, —(CH)ₙR^{a}—NR^{b}—CO—(CH)ₙR^{c}—, —(CH₂)ₙ—NR^{b}—CO—(CH)ₙR^{c}—, —(CH₂)ₙ—NR^{b}—CO—(CH₂)ₙ—, —(CH)ₙ—R^{a}—NR^{b}—SO₂—(CH₂)ₙ—, —(CH)ₙR^{a}—NR^{b}—SO₂—(CH)ₙR^{c}— and —(CH₂)ₙ—NR^{b}—SO₂—(CH)ₙR^{c}—, n is an integer selected from 0 to 6, R^{a} and R^{c} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl, R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, —C(═O)R^{a}, —C(═O)OR^{a}, —C(═O)NR^{a}R^{c} and —SO₂R^{a}, Y is either absent or selected from a group comprising —CH₂—, —CH₂CH₂—, —CH═CH—, C₃-C₆ cycloalkyl each of which is optionally substituted with a substituent selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl and A is selected from a group comprising Carbon and Nitrogen; a process for the preparation of compound of formula II,

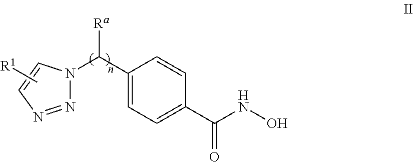

wherein, R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which may be optionally substituted with one or more substituents represented by R² wherein, R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl; n is an integer equal to 1 and R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl, said process comprising acts of, converting 1-Bromo-2-fluoro-4-methyl-benzene to an amine, coupling the amine with 4-Azidomethyl-benzoic acid methyl ester in the presence of copper iodide to obtain a triazole compound and reacting the triazole compound with a base to obtain compound of formula II, a pharmaceutical composition, comprising a compound of formula (I) along with pharmaceutically acceptable excipients(s) selected from a group comprising binders, disintegrants, diluents, lubricants, plasticizers, permeation enhancers and solubilizers; a method of inhibiting Histone deacetylase (HDAC), said method comprising contacting HDAC with a compound of formula (I), or prodrug of compound of formula (I) or pharmaceutical composition comprising compound of formula (I) optionally along with pharmaceutically acceptable excipients and a method of treating disease by HDAC inhibition, said method comprising administering biologically suitable amounts of compound of formula (I), prodrug of compound of formula(I) pharmaceutical composition comprising formula (I) optionally along with pharmaceutically acceptable excipients(s) to a subject in need thereof.

### BRIEF DESCRIPTION OF FIGURES

In order that the disclosure may be readily understood and put into practical effect, reference will now be made to exemplary embodiments as illustrated with reference to the accompanying figures. The figure together with a detailed description below, are incorporated in and form part of the specification, and serve to further illustrate the embodiments and explain various principles and advantages, in accordance with the present disclosure where:

FIG. 1: Oral pharmacokinetics of Example 13 and SAHA in male Balb/c mouse.

FIG. 2: Effect of the compound on tumor growth inhibition in A549 xenograft in nude mice

### DETAILED DESCRIPTION OF DISCLOSURE

The present disclosure is in relation to a compound of formula (I),

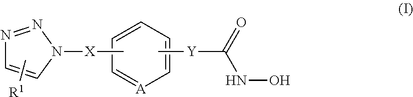

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein ;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylamino carbonyl, arylaminocarbonyl and heteroarylcarbonyl;

X is either absent or is selected from a group comprising cycloalkyl, —(CH₂)ₙ—, —(CH)ₙR^{a}—, —(CH₂)ₙ—NR^{b}—CO—(CH₂)ₙ—, —(CH)ₙR^{a}—NR^{b}—CO—(CH₂)ₙ—, —(CH)ₙR^{a}—NR^{b}—CO—(CH)ₙR^{c}—, —(CH₂)ₙ—NR^{b}—CO—(CH)ₙR^{c}—, —(CH₂)ₙ—NR^{b}—CO—(CH₂)ₙ—, —(CH)ₙR^{a}—NR^{b}—SO₂—(CH₂)ₙ—, —(CH)ₙR^{a}—NR^{b}—SO₂—(CH)ₙR^{c}— and —(CH₂)ₙ—NR^{b}—SO₂—(CH)ₙR^{c}—;

n is an integer selected from 0 to 6;

R^{a} and R^{c} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl;

R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, —C(═O)R^{a}, —C(═O)OR^{a}, —C(═O)NR^{a}R^{c} and —SO₂R^{a};
Y is either absent or selected from a group comprising —CH₂—, —CH₂CH₂—, —CH═CH—, C₃-C₆ cycloalkyl each of which is optionally substituted with a substituent selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl; and

A is selected from a group comprising Carbon and Nitrogen.

In another embodiment of the present disclosure, compounds of general formula (II),

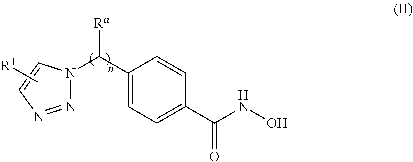

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

n is an integer equal to 1; and

R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl.

In still another embodiment of the present disclosure, compounds of general formula (III)

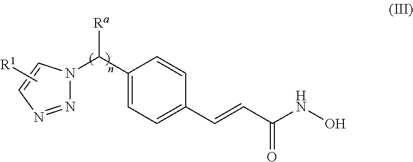

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein ;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

n is an integer equal to 1; and

R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl.

In yet another embodiment of the present disclosure, compound of general formula (IV)

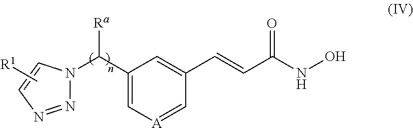

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein ;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

n is an integer equal to 1;

R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl; and

A is selected from a group comprising carbon and nitrogen.

In yet another embodiment of the present disclosure, compound of general formula (V)

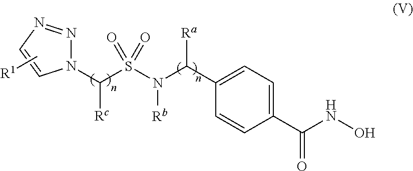

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylamino carbonyl, heteroarylcarbonyl, aryl and heteroaryl each of which is optionally substituted with one or more substituents represented as R² wherein ;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

n is an integer equal to 1;

R^{a} and R^{e} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl; and

R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, —C(═O)R^{a}, —C(═O)NR^{a}R^{c} and —SO₂R^{a}.

In yet another embodiment of the present disclosure, compound of general formula (VI)

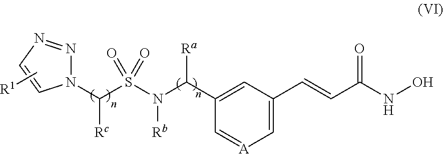

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which may be optionally substituted with one or more substituents represented as R² wherein ;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

n is an integer equal to 1;

R^{a} and R^{c} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl;

R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, C(═O)R^{a}, —C(═O)OR^{a}, —C(═O)NR^{a}R^{c} and —SO₂R^{a}; and A is selected from a group comprising Carbon and Nitrogen.

The present disclosure is also in relation to a process for the preparation of compound of formula II,

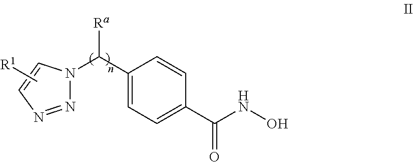

wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which may be optionally substituted with one or more substituents selected from R²;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

n is an integer equal to 1; and

R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl;

said process comprising acts of;
a) converting 1-Bromo-2-fluoro-4-methyl-benzene to an amine;
b) coupling the amine with 4-Azidomethyl-benzoic acid methyl ester in the presence of copper iodide to obtain a triazole compound; and
c) reacting the triazole compound with hydroxyl amine in presence of suitable base to obtain compound of formula II.

In yet another embodiment of the present disclosure, the base selected from group comprising sodium methoxide, sodium ethoxide and n-butyllithium, preferably sodium methoxide.

The present disclosure is also in relation to a pharmaceutical composition, comprising a compound of formula (I) along with pharmaceutically acceptable excipients(s) selected from a group comprising binders, disintegrants, diluents, lubricants, plasticizers, permeation enhancers and solubilizers.

In yet another embodiment of the present disclosure, compound of the compound of formula (I) is selected from a group comprising compounds of formula (II), formula (III), formula (IV), formula (V), and formula (VI).

In yet another embodiment of the present disclosure, said composition is in form selected from a group comprising tablet, capsule, powder, syrup, solution, aerosol and suspension.

The present disclosure is also in relation to a method of inhibiting Histone deacetylase (HDAC), said method comprising contacting HDAC with a compound of formula (I), or prodrug of compound of formula (I) or pharmaceutical composition comprising compound of formula (I) optionally along with pharmaceutically acceptable excipients.

The present disclosure is also in relation to a method of treating disease by HDAC inhibition, said method comprising administering biologically suitable amounts of compound of formula (I), prodrug of compound of formula(I) pharmaceutical composition comprising formula (I) optionally along with pharmaceutically acceptable excipients(s) to a subject in need thereof.

In yet another embodiment of the present disclosure, the compound of formula (I) is selected from a group comprising compounds of formula(II), formula (III), formula (IV), formula (V), and formula (VI).

In yet another embodiment of the present disclosure, the subject is an animal, including human beings.

Reference now will be made in detail to the embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment can be used on another embodiment to yield a still further embodiment. Thus, it is intended that the present disclosure cover such modifications and variations as come within the scope of the appended claims and their equivalents. Other objects, features and aspects of the present disclosure are disclosed in or are obvious from the following detailed description. It is to be understood by one of ordinary skill in the art that the present discussion is a description of exemplary embodiments only, and is not intended as limiting the broader aspects of the present disclosure.

Abbreviations and Definitions

The term “alkyl,” is intended to include both branched and straight-chain saturated aliphatic hydrocarbon groups having a specified number of carbon atoms. Exemplary alkyl groups of the disclosure have from 1 to 10 carbon atoms. Branched means a lower alkyl group such as methyl, ethyl or propyl, is attached to a linear alkyl chain. Non-limiting examples of suitable alkyl groups include methyl, ethyl, n-propyl, isopropyl, n-butyl, and t-butyl.

The term ‘cycloalkyl’ group refers to a cyclic alkyl group which may be mono, bicyclic or polycyclic. Exemplary cycloalkyl groups include cyclopropyl, cyclobutyl, cyclopentyl, cyclohexyl, cycloheptyl, cyclooctyl and the like. Unless otherwise specified, a cycloalkyl group typically has from 3 to about 10 carbon atoms. Typical bridged cycloalkyls include, but are not limited to, adamantyl, noradamantyl, bicyclo[1.1.0]butanyl, norboranyl(bicyclo[2.2.1]heptanyl), norbornenyl (bicyclo[2.2.1]heptanyl), norbornadienyl(bicyclo[2.2.1]heptadienyl), tricyclo[2.2.1]heptanyl, bicyclo[3.2.1]octanyl, bicyclo[3.2.1]octanyl, bicyclo[3.2.1]octadienyl, bicyclo[2.2.2]octanyl, bicyclo[2.2.2]octenyl, bicyclO[2.2.2]octadienyl, bicyclo[5.2.0]nonanyl, bicyclo[4.3.2]undecanyl, tricyclo[5.3.1.1]dodecanyl, and the like.

The term “cycloalkylalkyl” group is a (C₃-C₁₀)cycloalkyl-(C₁-C₁₀)alkyl group which may be mono or polycyclic. Exemplary cycloalkylalkyl groups include cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, cycloheptylmethyl, cycloheptylethyl, cyclooctylmethyl, cyclooctylethyl, cyclooctylpropyl, bicyclo[3.2.1]octanylmethyl, bicyclo[3.2.1]octanylmethyl, bicyclo[3.2.1]octadienyrmethyl, bicyclo[2.2.2]octanylmethyl and the like.

The term “heterocyclyl” is a non-aromatic saturated monocyclic or polycyclic ring system of about 5 to about 10 carbon atoms, having at least one hetero atom selected from O, S or N. Exemplary heterocyclyl groups include aziridinyl, pyrrolidinyl, piperdinyl, piperazinyl, morpholinyl, thiomorpholinyl, thiazolidinyl, 1,3-dioxolanyl, 1,4-dioxanyl and the like. The term “alkylaminoalkyl” is defined as the following representative examples and the like

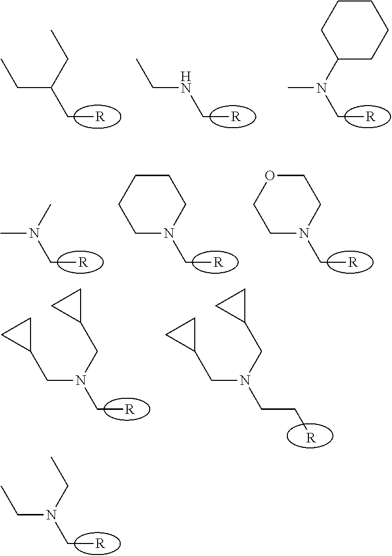

The term “alkoxy,” is intended to mean a chain of carbon atoms and is defined as ‘alkyl-O—’, wherein alkyl group is as defined above. The chains of carbon atoms of the alkoxy groups described and claimed herein are saturated, may be straight chain or branched. In a non-limiting example, “C₁-C₄ alkoxy” denotes an alkoxy group having carbon chain with from 1 to 4 carbon atoms, inclusive, straight chain or branched. Exemplary C₁-C₄ alkoxy groups include methoxy, ethoxy, propoxy, isopropoxy and the like.

As used herein, the term “aryl” means an aromatic or partially aromatic monocyclic or polycyclic ring system comprising about 6 to about 14 carbon atoms, preferably about 6 to about 10 carbon atoms. Non-limiting examples of suitable aryl groups include phenyl, naphthyl, 1, 2, 3, 4-Tetrahydro-naphthyl, and Indanyl.

The term “aryl alkyl” is the aryl—(C₁-C₁₀) alkyl group, wherein aryl and (C₁-C₁₀) alkyl groups are as defined above. Exemplary arylalkyl groups include benzyl, ethylphenyl, propylphenyl, butylphenyl, propyl-2-phenylethyl and the like.

The term “heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrrolyl, triazolyl, benzooxazolyl, benzothiazolyl and the like.

As used herein, the term “heteroarylalkyl” is the heteroaryl —(C₁-C₁₀) alkyl group, wherein heteroaryl and (C₁-C₁₀) alkyl groups are as defined above. Exemplary heteroarylalkyl groups include methylpyridine and the like.

The term “halogen” means fluoro, chloro, bromo or iodo groups.

The term ‘optionally substituted’ means that substitution is optional and therefore it is possible for the designated atom or molecule to be unsubstituted. In the event a substitution is desired, then such substitution means that any number of hydrogens on the designated atom is replaced with a selection from the indicated group, provided that the normal valency of the designated atom is not exceeded, and that the substitution results in a stable compound.

Pharmaceutically acceptable salts include base addition salts such as alkali metal salts like Li, Na, and K salts; alkaline earth metal salts like Ca and Mg, salts of organic bases such as lysine, arginine, guanidine, diethanolamine, α-phenylethylamine, benzylamine, piperidine, morpholine, pyridine, hydroxyethylpyrrolidine, hydroxyethylpiperidine, choline and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate, which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprising of other solvents of crystallization such as alcohols.

The term analog includes a compound, which differs from the parent structure by one or more C, N, O or S atoms. Hence, a compound in which one of the N atoms in the parent structure is replaced by an S atom is an analog of the former.

The term stereoisomer includes isomers that differ from one another in the way the atomsare arranged in space, but whose chemical formulas and structures are otherwise identical. Stereoisomers include enantiomers and diastereoisomers.

The term tautomers include readily interconvertible isomeric forms of a compound in equilibrium. The enol-keto tautomerism is an example.

The term polymorphs include crystallographically distinct forms of compounds with chemically identical structures.

The term pharmaceutically acceptable solvates includes combinations of solvent molecules with molecules or ions of the solute compound. The term derivative refers to a compound obtained from a compound according to formula (I), an analog, tautomeric form, stereoisomer, polymorph, hydrate, pharmaceutically acceptable salt or pharmaceutically acceptable solvate thereof, by a simple chemical process converting one or more functional groups, such as, by oxidation, hydrogenation, alkylation, esterification, halogenation, and the like. A term once described, the same meaning applies for it, throught the patent.

### LIST OF ABBREVIATIONS

mg—milligram

μg—microgram

ng nanogram

mL milliliter

μL microliter

mM—milli molar

μM—micro molar

nM—nano molar

m/z—mass/charge ratio

amu—atomic mass unit

msec—millisecond

h—hour

b.w.—body weight

v/v—volume ratio

CC—calibration curve

LLOQ—lower limit of quantification

ULLOQ—upper limit of quantification

Na₂EDTA—Disodium ethylene diamine tetra acetate

LC-MS/MS—Liquid Chromatography with tandem mass spectrometric detection

MRM—Multiple reaction monitoring

IS—Internal standard

r—Coefficient correlation

QC—quality control sample

% CV—percent coefficient of variation

STDV—standard deviation

PK—Pharmacokinetics

Cₘₐₓ—Concentration maximum

Tₘₐₓ—Time maximum

AUC₀ₜₒₜ—Area under curve 0 to time

AUC_{0toinf}—Area under curve 0 to infinity

AUCₗₐₛₜ—Area under curve 0 to last

AUCₑₓₜᵣₐₚ—Area under curve extrapolated

T_{1/2}—Half life

CL—Clearance

Vd—Volume of distribution

MRT—Mean retention time

AURC—last Area under recovered concentration 0 to last

SAHA—Suberoylanilide Hydroxamic Acid.

HDAC—Histone deacetylase.

The present disclosure provides a compound of formula (I),

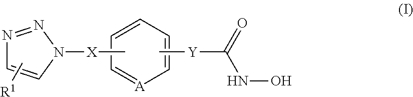

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein ;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

X is either absent or is selected from a group comprising cycloalkyl, —(CH₂)ₙ—, —(CH)ₙR^{a}—, —(CH₂)ₙ—NR^{b}—CO—(CH₂)ₙ—, —(CH)ₙR^{a}—NR^{b}—CO—(CH₂)ₙ—, —(CH)ₙR^{a}—NR^{b}—CO—(CH)ₙR^{c}—, —(CH₂)ₙ—NR^{b}—CO—(CH)ₙR^{c}—, —(CH₂)ₙ—NR^{b}—CO—(CH₂)ₙ—, —(CH)ₙR^{a}—NR^{b}—SO₂—(CH₂)ₙ—, —(CH)ₙR^{a}—NR^{b}—SO₂—(CH)ₙR^{c}— and —(CH₂)ₙ—NR^{b}—SO₂—(CH)ₙR^{c}—;

n is an integer selected from 0 to 6;

R^{a} and R^{c} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COON, alkoxycarbonyl, alkylaminocarbonyl, arylaminocarbonyl and heteroarylcarbonyl;
R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, —C(═O)R^{a}, —C(═O)OR^{a}, —C(═O)NR^{a}R^{c} and —SO₂R^{a};

Y is either absent or selected from a group comprising —CH₂—, —CH₂CH₂—, —CH═CH—, C₃-C₆ cycloalkyl each of which is optionally substituted with a substituent selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COON, alkoxycarbonyl, alkylaminocarbonyl, arylaminocarbonyl and heteroarylcarbonyl; and

A is selected from a group comprising Carbon and Nitrogen.

The present disclosure provides triazole derivatives of the general formula (I), having the general formula (II),

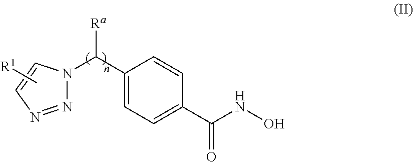

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein ;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

n is an integer equal to 1; and

R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl.

The present disclosure, provides compound of the general formula (I), having general formula (III)

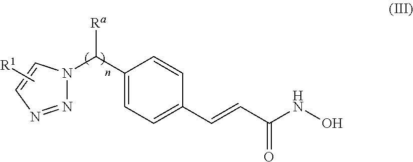

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

n is an integer equal to 1; and

R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl.

The present disclosure, provides triazole derivatives of the general formula (I), having the general formula (IV)

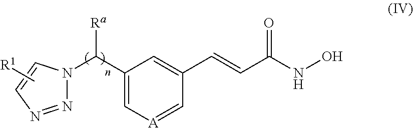

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein ;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylamino carbonyl, arylaminocarbonyl and heteroarylcarbonyl;

n is an integer equal to 1;

R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl; and

A is selected from a group comprising carbon and nitrogen.

The present disclosure, provides triazole derivatives of the general formula (I), having general formula (V)

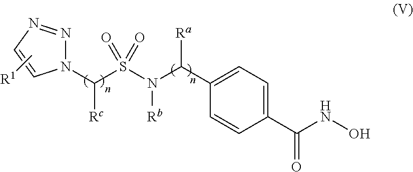

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein ;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

n is an integer equal to 1;

R^{a} and R^{c} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl; and

R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, —C(═O)OR^{a}, —C(═O)NR^{a}R^{c} and —SO₂R^{a}.

The present disclosure, provides triazole derivatives of the general formula (I), having general formula (VI)

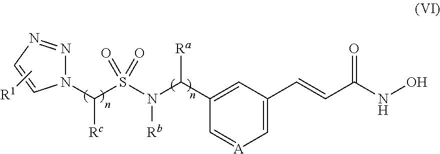

its derivatives, analogs, tautomeric forms, stereoisomers, polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which may be optionally substituted with one or more substituents represented as R² wherein;

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;

n is an integer equal to 1;

R^{a} and R^{c} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl;

R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, —C(═O)R^{a}, —C(═O)OR^{a}, —C(═O)NR^{a}R^{c} and —SO₂R^{a}; and

A is selected from a group comprising Carbon and Nitrogen.

Representative Compounds Include and Not Limiting to:
1. N-Hydroxy-4-(4-phenyl-[1,2,3]triazol-1-ylmethyl)-benzamide
2. 4-[4-(4-Fluoro-phenyl)-[1,2,3]triazol-1-ylmethyl]-N-hydroxy-benzamide
3. 4-[4-(2-Fluoro-phenyl)-[1,2,3]triazol-1-ylmethyl]-N-hydroxy-benzamide
4. N-Hydroxy-4-(4-pyridin-3-yl-[1,2,3]triazol-1-ylmethyl)-benzamide
5. 4-(4-Biphenyl-4-yl-[1,2,3]triazol-1-ylmethyl)-N-hydroxy-benzamide
6. N-Hydroxy-4-[4-(4-methoxy-phenyl)-[1,2,3]triazol-1-ylmethyl]-benzamide
7. N-Hydroxy-4-[4-(4-pyrrolidin-1-ylmethyl-phenyl)-[1,2,3]triazol-1-ylmethyl]-benzamide
8. N-Hydroxy-4-[4-(4-morpholin-4-ylmethyl-phenyl)-[1,2,3]triazol-1-ylmethyl]-benzamide
9. 4-[4-(4-Dimethylaminomethyl-phenyl)-[1,2,3]triazol-1-ylmethyl]-N-hydroxy-benzamide
10. (E)-3-{3-[4-(4-Dimethylamino-phenyl)-[1,2,3]triazol-1-ylmethyl]-phenyl}-N-hydroxy-acrylamide
11. 4-{4-[4-(2-Diethylamino-ethyl)-phenyl]-[1,2,3]triazol-1-ylmethyl}-N-hydroxy-benzamide
12. N-Hydroxy-4-{4-[4-(2-morpholin-4-yl-ethyl)-phenyl]-[1,2,3]triazol-1-ylmethyl}-benzamide
13. 4-[4-(4-Diethylaminomethyl-2-fluoro-phenyl)-[1,2,3]triazol-1-ylmethyl]-N-hydroxy-benzamide
14. 4-[4-(2-Fluoro-4-morpholin-4-ylmethyl-phenyl)-[1,2,3]triazol-1-ylmethyl]-N-hydroxy-benzamide
15. 4-[4-(2-Fluoro-4-pyrrolidin-l-ylmethyl-phenyl)-[1,2,3]triazol-1-ylmethyl]-N-hydroxy-benzamide
16. (E)-N-Hydroxy-3-[3-(4-phenyl[1,2,3]triazol-1-ylmethyl)-phenyl]-acrylamide
17. N-Hydroxy-3-[3-(4-pyridin-3-yl-[1,2,3]triazol-1-ylmethyl)-phenyl]-acrylamide
18. N-Hydroxy-3-{4-[4-(4-hydroxymethyl-phenyl)-[1,2,3]triazol-1-ylmethyl]-phenyl}-acrylamide
19. N-Hydroxy-3-{3-[4-(4-morpholin-4-ylmethyl-phenyl)-[1,2,3]triazol-1-ylmethyl]-phenyl}-acrylamide hydrochloride
20. N-Hydroxy-3-{3-[4-(4-pyrrolidin-1-ylmethyl-phenyl)-[1,2,3]triazol-1-ylmethyl]-phenyl}-acrylamide hydrochloride
21. 3-{3-[4-(4-Dimethylaminomethyl-phenyl)-[1,2,3]triazol-1-ylmethyl]-phenyl}-N-hydroxy-acrylamide hydrochloride
22. (E)-3-{3-[4-(4-Dimethylamino-phenyl)-[1,2,3]triazol-1-ylmethyl]-phenyl}-N-hydroxy-acrylamide
23. (E)-N-Hydroxy-3-[3-(4-phenyl-[1,2,3]triazol-1-ylmethyl)-phenyl]-acrylamide
24. N-Hydroxy-3-[3-(4-pyridin-3-yl-[1,2,3]triazol-1-ylmethyl)-phenyl]-acrylamide
25. N-Hydroxy-3-{3-[4-(4-morpholin-4-ylmethyl-phenyl)-[1,2,3]triazol-1-ylmethyl]-phenyl}-acrylamide hydrochloride
26. N-Hydroxy-3-{3-[4-(4-pyrrolidin-1-ylmethyl-phenyl)-[1,2,3]triazol-1-ylmethyl]-phenyl}-acrylamide hydrochloride
27. 3-{3-[4-(4-Dimethylaminomethyl-phenyl)-[1,2,3]triazol-1-ylmethyl]-phenyl}-N-hydroxy-acrylamide hydrochloride
28. 4-{[4-(4-Fluoro-phenyl)-[1,2,3]triazol-1-ylmethanesulfonylamino)-methyl}-N-hydroxy-benzamide
29. 4-{[4-(2-Fluoro-phenyl)-[1,2,3]triazol-1-ylmethanesulfonylamino]-methyl}-N-hydroxy-benzamide
30. N-Hydroxy-4-[(4-p-tolyl-[1,2,3]triazol-1-ylmethanesulfonylamino)-methyl]-benzamide
31. 4-{[4-(4-Dimethylamino-phenyl)-[1,2,3]triazol-1-ylmethanesulfonylamino]-methyl}-N-hydroxy-benzamide
32. N-Hydroxy-4-{[4-(4-methoxy-phenyl)-[1,2,3]triazol-1-ylmethanesulfonylamino]-methyl}-benzamide
33. N-Hydroxy-4-[(4-pyridin-3-yl-[1,2,3]triazol-1-ylmethanesulfonylamino)-methyl]-benzamide
34. N-Hydroxy-3-{3-[(4-phenyl-[1,2,31triazol-1-ylmethanesulfonylamino)-methyl]-phenyl}-acrylamide
35. 3-(3-{[4-(2-Fluoro-phenyl)-[1,2,3]triazol-1-ylmethanesulfonylamino]-methyl}-phenyl)-N-hydroxy-acrylamide

The present disclosure is provided by the examples given below, which are provided by the way of illustration only, and should not be considered to limit the scope of the disclosure. Variation and changes, which are obvious to one skilled in the art, are intended to be within the scope and nature of the disclosure, which are defined in the appended claims.

### EXAMPLES

Preparation of Intermediates:

### Example (a)

### Preparation of 1-Ethynyl-4-methoxy-benzene

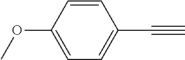

Step 1

To a solution of 4-Methoxy-benzaldehyde (4.0 g, 29.0 mmol) in dichloromethane (100 mL) were added carbon tetrabromide (19.4 g, 58.8 mmol) and triphenylphosphine (15.75 g, 58.8 mmol) in portions at 0° C. The reaction mixture was stirred at 25° C. over a period of 2 h. The resulting mixture was diluted with n-Hexane (200 mL) to obtain a triphenylphosphine oxide as a precipitate. The precipitate was filtered and solvent was evaporated under reduced pressure to obtain a crude product. The crude product was further purified by column chromatography to give 1-(2,2-Dibromo-vinyl)-4-methoxy-benzene as a off-white solid (7.0 g, 82.0%)

Step 2

To a solution of 1-(2,2-Dibromo-vinyl)-4-methoxy-benzene (4.0 g, 31.7 mmol) in dry THF was added n-butyl lithium (1.05 g, 16.5 mmol) at −78° C. over a period of 5 min. The reaction mixture was stirred at same temperature over a period of 60 min. Then resulting mixture was quenched with saturated ammonium chloride at −78° C., THF was evaporated under reduced pressure and the crude mixture was diluted with ethyl acetate (100 mL). The ethyl acetate layer was washed with water and dried over sodium sulphate. The solvents were evaporated under reduced pressure to give crude 1-Ethynyl-4-methoxy-benzene as light yellow oil (1.2 g, 66%).

The following compounds were synthesized by using the procedure disclosed above or analogous to the above procedure

| Preparation | | |
|---|---|---|
| No. | Structure | IUPAC name |
| (b) | 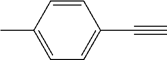 | 1-Ethynyl-4-methyl- benzene |
| (c) | 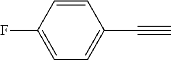 | 1-Ethynyl-4-fluoro- benzene |
| (d) | 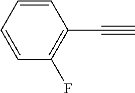 | 1-Ethynyl-2-fluoro- benzene |
| (e) | 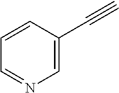 | 3-Ethynyl-pyridine |
| (f) | 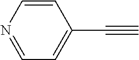 | 4-Ethynyl-pyridine |
| (g) | 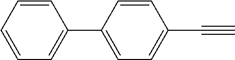 | 4-Ethynyl-biphenyl |

### Example (h)

### Preparation of 4-(4-Ethynyl-benzyl)-morpholine

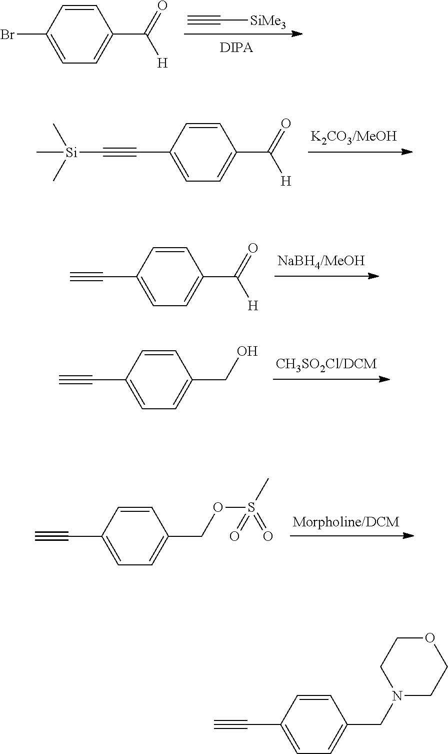

Step 1: To a solution of 4-bromobenzaldehyde (10.0 g, 54.04 mmol) in diisopropylamine (600 mL) were added bistriphenylphosphine palladium (II) chloride (380 mg, 0.54 mmol) and CuI (205 mg, 1.08 mmol). The reaction mixture was degassed for 20 min. Then the reaction mixture was cooled to ice temperature and trimethyl silylacetalide (11.2 mL, 81.06 mmol) was added drop wise at same temperature for 30 min and it was refluxed over a period of 3 h. Diisopropylamine was evaporated under reduced pressure and the residue was diluted with ethyl acetate (1000 mL). The ethyl acetate layer was washed with 1N Hydrochloric acid (2×100 mL), saturated sodium bicarbonate (1×100 mL) and water (2×200 mL). Organic layer was dried over sodium sulphate and it was evaporated under reduced pressure to obtain crude product. The crude product was further purified by column chromatography to give 4-Trimethylsilanylethynyl-benzaldehyde as a colorless solid (8.5 g, 80%).

Step 2: To a solution of 4-Trimethylsilanylethynyl-benzaldehyde (4.0 g, 19.7 mmol) in methanol (50 mL) was added K₂CO₃ (275 mg, 1.97 mmol) at 25° C. The reaction mixture stirred at same temperature over a period of 60 min. Methanol was evaporated to the half volume at 35° C. and it was diluted with ethyl acetate (500 mL). The organic layer washed with water (2×100 mL) and dried over sodium sulphate and it was evaporated under reduced pressure to obtain crude product. The crude product was further purified by column chromatography to give 4-Ethynyl-benzaldehyde as a light yellow solid (1.8 g, 72%).

Step 3: To a solution of 4-Ethynyl-benzaldehyde (1.8 g, 13.8 mmol) in methanol (40 mL) was added NaBH₄ (1.04 g, 27.1 mmol) at 0° C. over a period of 5 min. The reaction mixture was allowed to stir at 25° C. over a period of 60 min. The reaction mixture quenched with saturated ammonium chloride and the solvent was evaporated under reduced pressure. The crude product was diluted with ethyl acetate (200 mL) washed with water (2×50 mL) dried over sodium sulphate and it was evaporated under reduced pressure to give (4-Ethynyl-phenyl)-methanol as a light yellow oil (1.1 g, 61%).

Step 4: To a solution of (4-Ethynyl-phenyl)-methanol (1.6 g, 12.1 mmol) in dichloromethane (40 mL) were added triethylamine (5.05 mL, 36.3 mmol) followed by methane sulfonyl chloride at 0° C. The reaction mixture was stirred at 25° C. over a period of 12 h. The resulting reaction mixture was diluted with dichloromethane (60 mL), washed with water (2×50 mL), saturated brine (1×50 mL) and dried over sodium sulphate. The solvent was evaporated under reduced pressure to give Methanesulfonic acid 4-ethynyl-benzyl ester as reddish viscous oil (2.3 g, 90.5%).

Step 5: To a solution of Methanesulfonic acid 4-ethynyl-benzyl ester (2.3 g, 10.9 mmol) in dichloromethane (40 mL) were added triethylamine (3.03 mL, 21.2 mmol) followed by morpholine (2.36 mL, 27.3 mmol) at ice temperature. The reaction mixture was stirred at 25° C. over a period of 12 h. The resulting reaction mixture was diluted with dichloromethane (500 mL), washed with water (3×100 mL), brine (1×100 mL) and dried over sodium sulphate. The crude product obtained was further purified by column chromatography to obtain 4-(4-Ethynyl-benzyl)-morpholine as a light yellow solid (2.0 g, 91%).

¹H NMR (300 MHz, DMSO-d₆) δ (ppm): 2.31-2.34 (m, 4H), 3.46 (s, 1H), 3.54-3.57 (m, 4H), 4.14 (s, 1H), 7.31 (d, J=7.8 Hz, 2H), 7.43 (d, J=8.1 Hz, 2H).

The following compounds were synthesized by using the procedure disclosed above or analogous to the above procedure

| Preparation No. | Structure | Analytical data |
|---|---|---|
| (i) | 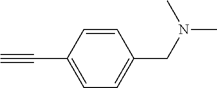 | ¹H NMR (300 MHz, CDCl₃) δ (ppm): 2.52 (s, 6H), 3.07 (s, 1H), 3.44 (s, 2H), 7.27-7.30 (m, 2H), 7.45-7.48 (m, 2H). |
| (j) | 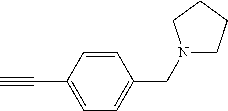 | ¹H NMR (300 MHz, CDCl₃) δ (ppm): 1.78-1.83 (m, 4H), 2.49-2.53 (m, 4H), 3.03 (s, 1H), 3.62 (s, 2H), 7.28-7.31 (m, 2H), 7.43-7.46 (m, 2H). |

### Example (k)

### Preparation of diethyl-(4-ethynyl-2-fluoro-benzyl)-amine

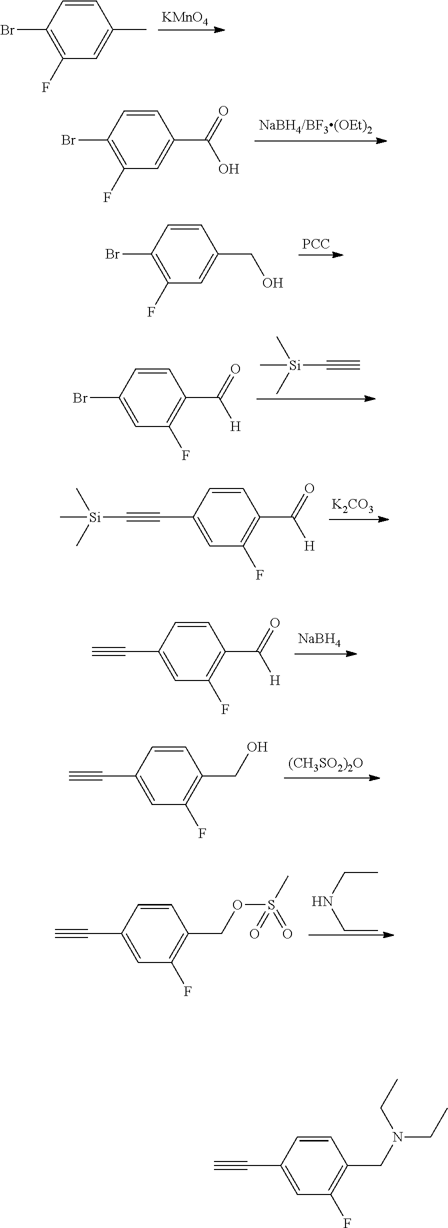

Step 1: To a solution of 1-bromo-2-fluoro-4-methyl-benzene (20.0 g, 0.10 mol) in 1:1 ratio of pyridine: water (200 mL) was added potassium permanganate (66.0 g, 0.42 mmol) portion wise at 90° C. and the reaction mixture was stirred at 90° C. over a period of 3 h. The resulting reaction mixture was allowed to reach room temperature and filtered trough celite pad. The celite pad was washed with 3N sodium hydroxide (500 mL) and water (400 mL). Then ethanol was removed under reduced pressure and residue was acidified (pH=2) with 6N hydrochloric acid to obtained white precipitate. The precipitate obtained was filtered and dried to give 4-bromo-3-fluoro-benzoic acid as white solid (17.0 g, 73%).

Step 2: To a suspension of sodium borohydride (10.4 g, 0.27 mol) in tetrahydrofuran (200 mL) was added boron trifluoride etherate (44.3 mL, 0.36 mol) followed by 4-bromo-3-fluoro-benzoic acid (10.0 g, 0.04 mol) in THF (200 mL) at ice temperature. The mixture was allowed to stir at room temperature over a period of 2 h. The resulting reaction mixture was quenched with methanol and methanol was removed under reduced pressure. The residue obtained upon evaporation of methanol was diluted with ethyl acetate (1.0 L), washed with water (700 mL), dried over sodium sulphate and concentrated to give (4-bromo-3-fluoro-phenyl)-methanol as off-white solid (7.9 g, 84%).

Step 3: To a solution of (4-bromo-3-fluoro-phenyl)-methanol (7.9 g, 38.5 mmol) in dichloromethane (160.0 mL) was added sodium acetate (940 mg, 11.5 mmol) followed by pyridinium chlorochromate (10.8 g, 50.0 mmol) at room temperature. The reaction mixture was stirred at room temperature under light protection over a period of 2 h. The resulting reaction mixture was diluted with ethyl acetate (1.0 L) and filtered through celite pad. The filtrate obtained was washed with aqueous sodium bicarbonate (600 mL), water (600 mL) and dried over sodium sulphate. The crude product obtained upon evaporation of the solvent was further purified by column chromatography to give 4-bromo-2-fluoro-benzaldehyde as white solid (5.0 g, 63%).

Step 4: To a solution of 4-bromo-2-fluoro-benzaldehyde (18.0 g, 89.5 mmol) in diisopropylamine (360 mL) were added bistriphenylphosphine palladium (II) chloride (3.1 g, 4.4 mmol) and CuI (1.79 g, 8.9 mmol). The reaction mixture was degassed for 20 min. Then the reaction mixture was cooled to ice temperature and trimethyl silylacetalide (13.1 g, 134.4 mmol) was added drop wise at same temperature for 60 min and it was refluxed over a period of 3 h. The reaction mixture was diluted with ethyl acetate (400 mL) and filtered through celite pad. The crude product obtained upon evaporation of volatiles was again diluted with ethyl acetate (1.5 L). The ethyl acetate layer was washed with water (3×800 mL), dried over sodium sulphate and it was evaporated under reduced pressure to obtain crude product. The crude product was further purified by column chromatography to give 2-fluoro-4-trimethylsilanylethynyl-benzaldehyde as a pale yellow solid (13.0 g, 68%).

Step 5: To a solution of 2-fluoro-4-trimethylsilanylethynyl-benzaldehyde (13.0 g, 59.3 mmol) in methanol (100 mL) was added K₂CO₃ (492 mg, 3.5 mmol) at 25° C. The reaction mixture stirred at same temperature over a period of 60 min. Methanol was evaporated to the half volume at 35° C. and it was diluted with ethyl acetate (500 mL). The organic layer washed with water (2×100 mL) and dried over sodium sulphate and it was evaporated under reduced pressure to obtain crude product. The crude product was further purified by column chromatography to give 4-ethynyl-2-fluoro-benzaldehyde as a light yellow solid (6.5 g, 81%).

Step 6: To a solution of 4-ethynyl-2-fluoro-benzaldehyde (6.5 g, 47.7 mmol) in isopropyl alcohol (60 mL) was added NaBH₄ (1.62 g, 43.0 mmol) at ice temperature over a period of 10 min. The reaction mixture was allowed to stir at 25° C. over a period of 60 min. The reaction mixture quenched with saturated ammonium chloride and the solvent was evaporated under reduced pressure. The crude product was diluted with ethyl acetate (200 mL) washed with water (2×50 mL) dried over sodium sulphate and it was evaporated under reduced pressure to give (4-ethynyl-2-fluoro-phenyl)-methanol as a light yellow solid (4.0 g, 56%).

Step 7: To a solution of (4-ethynyl-2-fluoro-phenyl)-methanol (4.0 g, 26.8 mmol) in dichloromethane (40 mL) were added pyridine (5.4 mL, 67.1 mmol) followed by methane sulfonic anhydride at ice temperature. The reaction mixture was stirred at 25° C. over a period of 2 h. The resulting reaction mixture was diluted with dichloromethane (100 mL), washed with water (2×50 mL), saturated brine (50 mL) and dried over sodium sulphate. The solvent was evaporated under reduced pressure to give methanesulfonic acid 4-ethynyl-2-fluoro-benzyl ester as reddish viscous oil (4.0 g, 90.5%).

Step 8: To a solution of methanesulfonic acid 4-ethynyl-2-fluoro-benzyl ester (1.2 g, 5.0 mmol) in acetonitrile (12 mL) were added triethylamine (1.4 mL, 12.0 mmol) followed by diethyl amine (1.3 mL, 13.0 mmol) at room temperature. The reaction mixture was stirred at 80° C. over a period of 1 h. The resulting reaction mixture was diluted with ethyl acetate (300 mL), washed with water (3×100 mL) and dried over sodium sulphate. The crude product obtained was further purified by column chromatography to obtain diethyl-(4-ethynyl-2-fluoro-benzyl)-amine as yellow color oil (830 mg, 83%).
¹H NMR (300 MHz, DMSO-d₆) δ (ppm): 0.96 (t, J=7.2 Hz, 6H), 2.41-2.50 (m, 4H), 3.54 (s, 2H), 4.43 (s, 1H), 7.16-7.23 (m, 2H), 7.48 (t, J=7.5 Hz, 1H).

The following compounds were synthesized by using the procedure disclosed above or analogous to the above procedure

| | | |
|---|---|---|
| (l) | 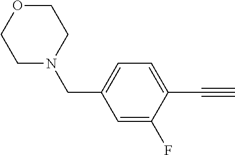 | ¹H NMR (300 MHz, DMSO-d₆) δ (ppm): 1.67-1.72 (m, 4H), 2.40-2.44 (m, 4H), 3.59 (s, 2H), 4.44 (s, 1H), 7.15-7.23 (m, 2H), 7.48 (t, J = 7.5 Hz, 1H). |
| (m) | 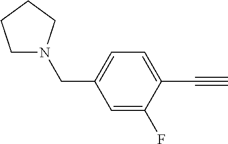 | ¹H NMR (300 MHz, DMSO-d₆) δ (ppm): 1.67-1.72 (m, 4H), 2.40-2.44 (m, 4H), 3.59 (s, 2H), 4.44 (s, 1H), 7.15-7.23 (m, 2H), 7.48 (t, J = 7.5 Hz, 1H). |

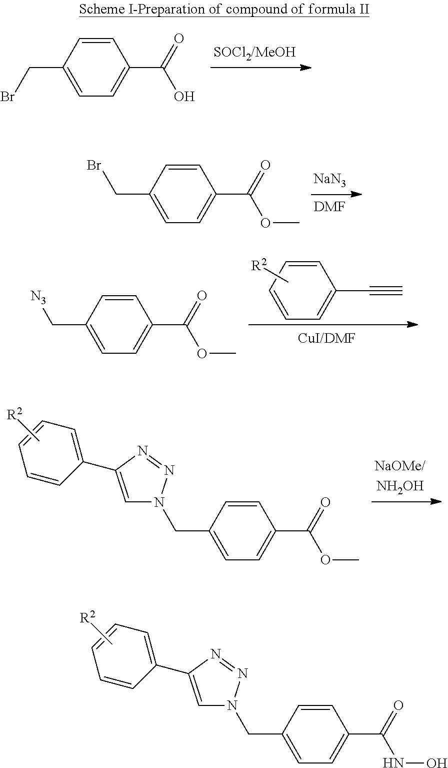

### Example 1

### Synthesis of 4-[4-(4-Dimethylamino-phenyl)-2, 3-dihydro[1,2,3]triazol-1-ylmethyl]-N-hydroxy-benzamide

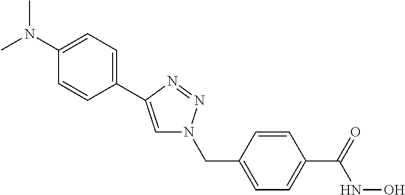

### Step 1: Preparation of 4-Bromomethyl-benzoic acid methyl ester

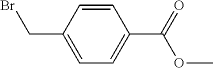

To a solution of 4-Bromomethyl-benzoic acid (10 g, 46 mmol) in methanol (80 mL) was added thionyl chloride (13.7 mL, 186 mmol) at 25° C. and the reaction mixture was stirred at same temperature over a period of 12 h. Then the solvent was evaporated under reduced pressure to obtain 4-Bromomethyl-benzoic acid methyl ester as yellow oil (9.6 g, 93.2%).

### Step 2: Preparation of 4-Azidomethyl-benzoic acid methyl ester

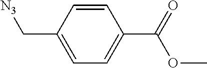

To a solution of 4-Bromomethyl-benzoic acid methyl ester (6.0 g, 28.0 mmol) in DMF (60 mL) was added sodium azide (3.6 g, 56.7 mmol) at 25° C. The reaction mixture was stirred at 80° C. over a period of 3 h. The resulting mixture was diluted with ethyl acetate and washed with water, brine and dried over sodium sulfate. The solvent was evaporated under reduced pressure to obtain 4-Azidomethyl-benzoic acid methyl ester as colorless solid (3.5 g, 64%).

### Step 3: Preparation of 4-[4-(4-Dimethylamino-phenyl)-[1,2,3]triazol-1-ylmethyl]-benzoic acid methyl ester

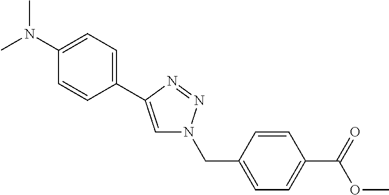

To a solution of 4-Azidomethyl-benzoic acid methyl ester (300 mg, 14.3 mmol) in DMF (6.0 mL) were added copper iodide (138 mg, 7.1 mmol), cat. sodium ascorbate, N-ethyl diisopropyl amine (0.48 mL, 29.0 mmol) and (4-Ethynyl-phenyl)-dimethyl-amine (227 mg, 15.7 mmol) at 25° C. The reaction mixture was stirred at same temperature over a period of 12 h. Then the resulting mixture was quenched with aqueous ammonia and diluted with ethyl acetate. The organic layer washed with water, brine and dried over sodium sulfate. The residue obtained upon evaporation of the volatiles was purified by column chromatography to give 4-[4-(4-Dimethylamino-phenyl)-[1,2,3]triazol-1-ylmethyl]-benzoic acid methyl ester as a colorless solid (200 mg, 41.92%).

### Step 4: Preparation of 4-[4-(4-Dimethylamino-phenyl)-2,3-dihydro-[1,2,3]triazol-1-ylmethyl]-N-hydroxy-benzamide

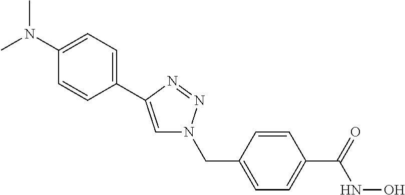

To a suspension of hydroxyl amine hydrochloride (3.1 g, 450 mmol) in methanol (30 mL) was added sodium methoxide (3.6 g, 670 mmol) at ice temperature and the suspension was stirred at ice temperature over a period of 30 min. To the above suspension 4-[4-(4-Dimethylamino-phenyl)-[1,2,3]triazol-1-ylmethyl]-benzoic acid methyl ester (1.5 g, 4.5 mmol) in methanol:dichloromethane (4:1, 10 mL)) was added drop wise at −20° C. The reaction temperature was allowed to reach 25° C. and stirred at same temperature over a period of 3 h. The resulting reaction mixture was acidified with acetic acid and the solvents were removed under reduced pressure to obtain crude colorless solid. The crude solid was further purified by column chromatography to give 4-[4-(4-Dimethylamino-phenyl)-2,3-dihydro-[1,2,3]triazol-1-ylmethyl]-N-hydroxy-benzamide as off-white solid (380 mg, 25%).

¹H NMR (300 MHz, DMSO-d₆) δ (ppm): 2.92 (s, 6H), 5.65 (s, 2H), 6.76 (d, J=8.7 Hz, 2H), 7.38 (d, J=7.8 Hz, 2H), 7.64 (d, J=8.7 Hz, 2H), 7.75 (d, J=7.8 Hz, 2H), 8.43 (s, 1H).

LCMS (ESI) m/z: 338 ([M+H⁺).

The following compounds were synthesized by using the procedure disclosed above or analogous to the above procedure

| Ex. No. | Structure | Analytical data |
|---|---|---|
| 2 | 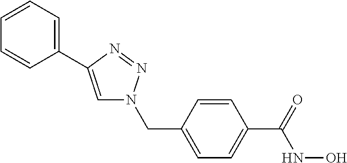 | ¹H NMR (300 MHz, DMSO-d₆) δ (ppm): 5.70 (s, 2H), 7.32-7.46 (m, 5H), 7.75 (d, J = 7.8 Hz, 2H), 7.84 (d, J = 8.1 Hz, 2H), 8.66 (s, 1H), 9.06 (s, 1H), 11.23 (s, 1H). LCMS (ESI) m/z: 294.8 ([M + H]⁺). |
| 3 | 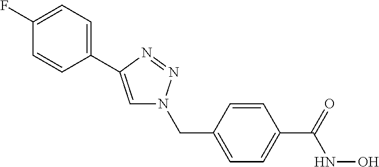 | ¹H NMR (300 MHz, DMSO-d₆) δ (ppm): 5.70 (s, 2H), 7.25-7.38 (m, 2H), 7.42 (d, J = 7.2 Hz, 2H), 7.76 (d, J = 8.4 Hz, 2H), 7.85-8.14 (m, 2H), 8.66 (s, 1H). LCMS (ESI) m/z: 313 ([M + H]⁺). |
| 4 | 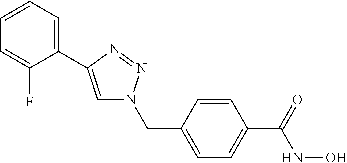 | ¹H NMR (300 MHz, DMSO-d₆) δ (ppm): 5.74 (s, 2H), 7.30-7.37 (m, 2H), 7.41 (d, J = 7.8 Hz, 3H), 7.74 (d, J = 8.1 Hz, 2H), 8.13 (t, J = 7.2 Hz, 1H), 8.58 (d, J = 3.6 Hz, 1H), 9.05 (s, 1H), 11.22 (s, 1H). LCMS (ESI) m/z: 312.9 ([M + H]⁺). |
| 5 | 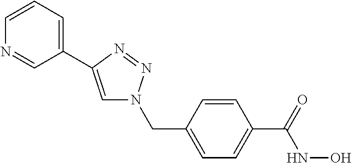 | ¹H NMR (300 MHz, DMSO-d₆) δ (ppm): 5.78 (s, 2H), 7.42 (d, J = 8.4 Hz, 2H), 7.77 (d, J = 8.1 Hz, 2H), 7.89-7.94 (m, 1H), 8.71-8.77 (m, 2H), 8.95 (s, 1H), 9.26 (s, 1H), 10.2 (bs, 1H), 11.4 (bs, 1H). LCMS (ESI) m/z: 295.9 ([M + H]⁺). |
| 6 | 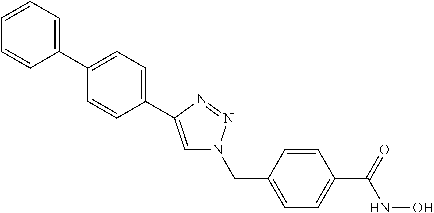 | ¹H NMR (300 MHz, DMSO-d₆) δ (ppm): 5.72 (s, 2H), 7.35-7.50 (m, 5H), 7.70-7.77 (m, 6H), 7.93-7.96 (m, 2H), 8.72 (s, 1H), 9.06 (bs, 1H), 11.24 (s, 1H). LCMS (ESI) m/z: 371.0 ([M + H]⁺). |
| 7 | 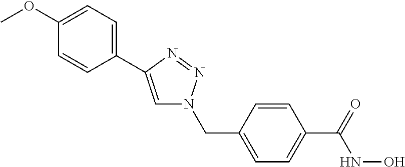 | ¹H NMR (300 MHz, DMSO-d₆) δ (ppm): 3.78 (s, 3H), 5.68 (s, 2H), 7.00 (d, J = 6.9 Hz, 2H), 7.74-7.78 (m, 2H), 7.97 (d, J = 6.9 Hz, 2H), 8.56 (s, 1H). LCMS (ESI) m/z: 325.0 ([M + H]⁺). |
| 8 | 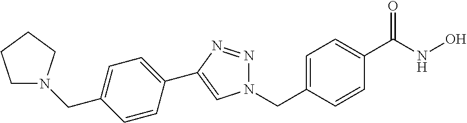 | ¹H NMR (300 MHz, DMSO-d₆) δ (ppm): 1.86-2.08 (m, 4H), 2.98-3.06 (m, 2H), 3.25-3.56 (m, 2H), 4.34 (d, J = 6.0 Hz, 2H), 5.72 (s, 2H), 7.40 (d, J = 8.1 Hz, 2H), 7.68 (d, J = 8.4 Hz, 2H), 7.76 (d, J = 8.1 Hz, 2H), 7.92 (d, J = 8.1 Hz, 2H), 8.74 (s, 1H). LCMS (ESI) m/z: 378.2 ([M + H]⁺). |
| 9 | 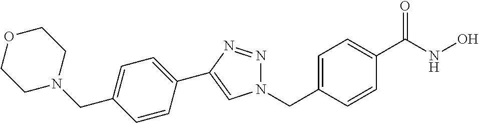 | ¹H NMR (300 MHz, DMSO-d₆) δ (ppm): 3.06-3.23 (m, 4H), 3.78-3.94 (m, 2H), 4.33 (d, J = 3.6 Hz, 2H), 5.72 (s, 2H), 7.40 (d, J = 8.1 Hz, 2H), 7.70 (d, J = 7.8 Hz, 2H), 7.76 (d, J = 7.8 Hz, 2H), 7.92 (d, J = 7.8 Hz, 2H), 8.75 (s, 1H). LCMS (ESI) m/z: 394.1 ([M + H]⁺). |
| 10 | 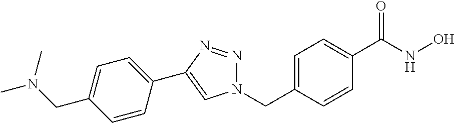 | ¹H NMR (300 MHz, DMSO-d₆) δ (ppm): 2.68 (s, 3H), 2.69 (s, 3H), 4.28 (d, J = 4.2 Hz, 2H), 5.72 (s, 2H), 7.40 (d, J = 7.5 Hz, 2H), 7.63 (d, J = 7.5 Hz, 2H), 7.77 (d, J = 7.5 Hz, 2H), 7.93 (d, J = 7.8 Hz, 2H), 8.75 (s, 1H), 10.70 (s, 1H), 11.25 (s, 2H). LCMS (ESI) m/z: 352.2 ([M + H]⁺). |
| 11 | 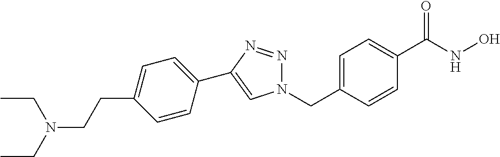 | ¹H NMR (300 MHz, DMSO-d₆) δ (ppm): 1.24-1.26 (m, 6H), 3.02-3.06 (m, 2H), 3.16-3.29 (m, 6H), 5.70 (s, 2H), 7.38-7.42 (m, 4H), 7.75-7.83 (m, 4H), 8.66 (s, 1H), 10.17 (bs, 1H) 11.26 (bs, 1H). |
| 12 | 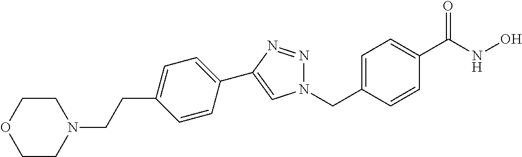 | ¹H NMR (300 MHz, DMSO-d₆) δ (ppm): 3.07-3.17 (m, 4H), 3.33-3.39 (m, 1H), 3.47-3.60 (m, 4H), 3.73-3.81 (m, 2H), 3.98-4.01 (m, 2H), 5.70 (s, 2H), 7.34-7.42 (m, 4H), 7.75-7.83 (m, 4H), 8.65 (s, 1H), 10.90 (bs, 1H) 11.25 (s, 1H). |
| 13 | 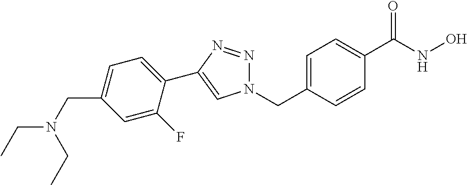 | ¹H NMR (300 MHz, DMSO-d₆) δ (ppm): 1.25 (t, J = 7.2 Hz, 6H), 3.04-3.08 (m, 4H), 4.34 (d, J = 5.4 Hz, 2H), 5.76 (s, 2H), 7.41 (d, J = 8.4 Hz, 2H), 7.54- 7.70 (m, 2H), 7.76 (d, J = 8.1 Hz, 2H), 8.20 (t, J = 8.1 Hz, 1H), 8.66 (d, J = 3.6 Hz, 1H), 10.40 (bs, 1H), 11.26 (bs, 1H). LCMS (ESI) m/z: 398.1 ([M + H]+). |
| 14 | 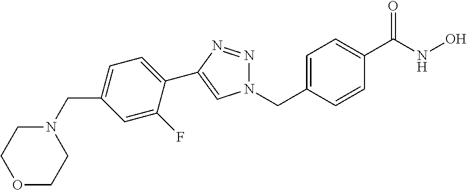 | ¹H NMR (300 MHz, DMSO-d₆) δ (ppm): 1.87-1.91 (m, 2H), 1.94-2.02 (m, 2H), 3.02-3.10 (m, 2H), 3.36-3.39 (m, 2H), 4.38 (d, J = 6.0 Hz, 2H), 5.76 (s, 2H), 7.42 (d, J = 8.4 Hz, 2H), 7.52 (d, J = 1.2 Hz, 1H), 7.54- 7.71 (m, 1H), 7.75 (d, J = 8.4 Hz, 2H), 8.18 (t, J = 8.1 Hz, 1H), 8.64 (s, 1H), 10.99 (bs, 1H), 11.21 (bs, 1H). LCMS (ESI) m/z: 396.1 ([M + H]+). |
| 15 | 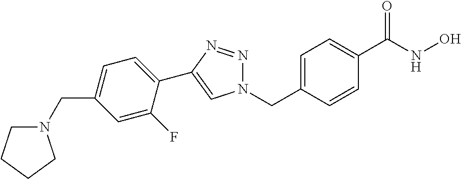 | ¹H NMR (300 MHz, DMSO-d₆) δ (ppm): 3.07-3.14 (m, 2H), 3.24-3.28 (m, 2H), 3.56-3.66 (m, 2H), 3.76-3.96 (m, 2H), 4.38 (bs, 2H), 5.76 (s, 2H), 7.41 (d, J = 8.1 Hz, 2H), 7.52 (d, J = 8.1 Hz, 1H) 7.66-7.70 (m, 1H), 7.75 (t, J = 8.1 Hz, 2H), 8.20 (t, J = 8.4 Hz, 1H), 8.66 (d, J = 3.6 Hz, 1H), 11.14 (bs, 1H), 11.24 (bs, 1H). LCMS (ESI) m/z: 412.1 ([M + H]+). |

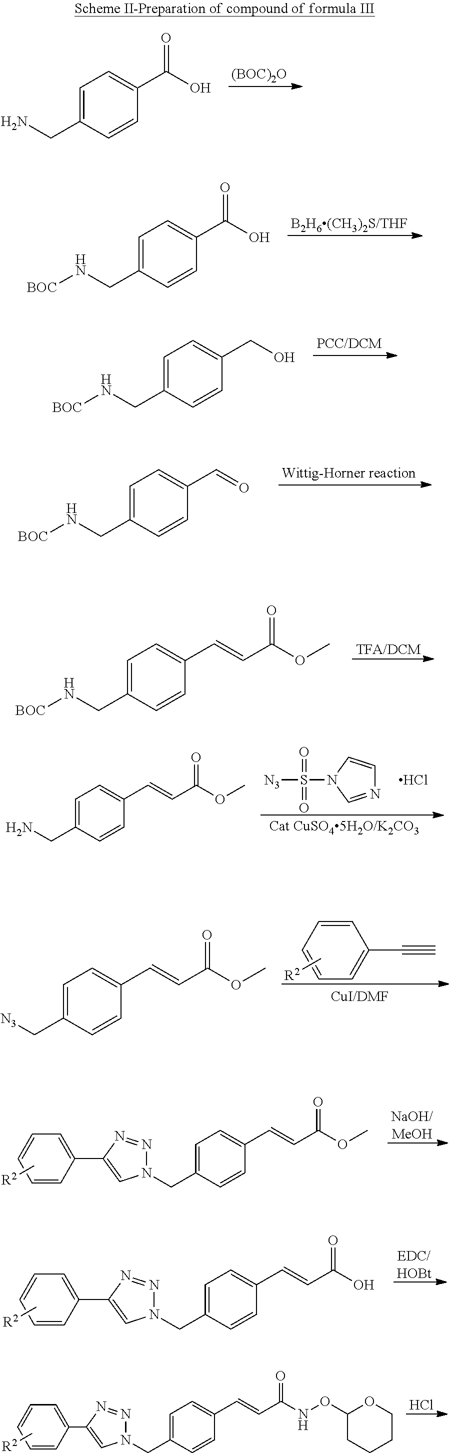

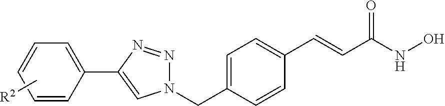

### Example 16

### Synthesis of N-Hydroxy-3-{4-[4-(4-pyrrolidin-1-ylmethyl-phenyl)-[1,2,3]triazol -1-ylmethyl]-phenyl}-acrylamide

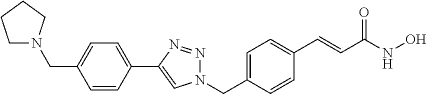

### Step 1: Preparation of 4-(tert-Butoxycarbonylamino-methyl)-benzoic acid

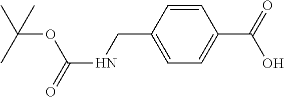

To a solution of 4-Aminomethyl-benzoic acid (5.0 g, 33.1 mmol) in 1,4-dioxane (60.0 mL) were added 1M sodium hydroxide (30 mL), BOC-anhydride (6.8 mL, 29.8 mmol) drop wise at ice temperature and it was stirred at same temperature over a period of 60 min. The resulting mixture acidified (P^{H}=5.0) with 1.5 N hydrochloric acid and extracted with ethyl acetate (3×100 mL), dried over sodium sulphate. Volatiles were evaporated under reduced pressure to obtain 4-(tert-Butoxycarbonylamino-methyl)-benzoic acid as a colourless solid (5.0 g, 60%).

### Step 2: Preparation of (4-Hydroxymethyl-benzyl)-carbamic acid tert-butyl ester

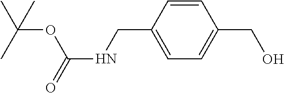

To a solution of 4-(tert-Butoxycarbonylamino-methyl)-benzoic acid (5.0 g, 19.7 mmol) in dry THF (50 mL) was added BMS (6.4 mL, 78.9 mmol) at ice temperature and it was stirred at 25° C. over a period of 12h. The resulting mixture was quenched with water and solvent was evaporated under reduced pressure to obtain crude mass. The crude mass was diluted with ethyl acetate (500 mL), washed with water (2×150 mL) and dried over sodium sulphate. The residue obtained upon evaporation of volatiles was purified by column chromatography to give (4-Hydroxymethyl-benzyl)-carbamic acid tert-butyl ester as an off-white solid (2.5 g, 53%).

### Step 3: Preparation of (4-Formyl-benzyl)-carbamic acid tert-butyl ester

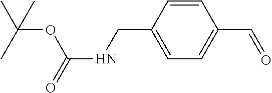

To a solution of (4-Hydroxymethyl-benzyl)-carbamic acid tert-butyl ester (4.5 g, 18.2 mmol) in dichloromethane (45 ml) were added PCC (4.07 g, 18 2 mmol), sodium acetate (0.26 g, 3.2 mmol) and the mixture was stirred at 25° C. over a period of 60 min. The resulting mixture was diluted with ethyl acetate (200 mL) and it was stirred for 30 min. Then the reaction mixture filtered through Buchner funnel, filtrate was washed with water (2×50 mL) and dried over sodium sulphate. The solvent was evaporated under reduced pressure to obtain (4-Formyl-benzyl)-carbamic acid tert-butyl ester as a pale yellow solid (3.0 g, 70%).

### Step 4: Preparation of 3-[4-(tert-Butoxycarbonylamino-methyl)-phenyl]-acrylic acid methyl ester

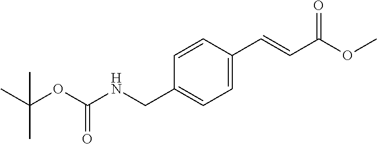

To a solution of trimethyl phosphino acetate (4.64 mL, 23.5 mmol) in dry DMF (20 mL) was added potassium tert-butoxide (2.14 g, 19.13 mmol) at 0° C. and the mixture was stirred at same temperature over a period of 15 min. To the above mixture a solution of (4-Formyl-benzyl)-carbamic acid tert-butyl ester (3.0 g, 12.75 mmol) in DMF (10 mL) was added drop wise at ice temperature. The reaction mixture was stirred at ice temperature over a period of 45 min. The resulting reaction mixture diluted with ethyl acetate, washed with water, brine and dried over sodium sulfate. The residue obtained upon evaporation of volatiles was purified by column chromatography to give 3-[4-(tert-Butoxycarbonylamino-methyl)-phenyl]-acrylic acid methyl ester as colourless solid (1.5 g, 40%).

### Step 5: Preparation of trifluoro acetate of 3-(4-Aminomethyl-phenyl)-acrylic acid methyl ester

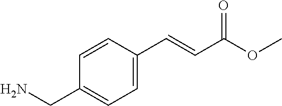

To a solution of 3-[4-(tert-Butoxycarbonylamino-methyl)-phenyl]-acrylic acid methyl ester (1.5 g, 5.14 mmol) in dichloromethane (5.2 mL) was added trifluoroacetic acid (5.2 mL) drop wise at ice temperature. The residue obtained upon evaporation of volatiles was washed with ether to give trifluoro acetate of 3-(4-Aminomethyl-phenyl)-acrylic acid methyl ester as colourless solid (0.6 g, 61%).

### Step 6: Preparation of 3-(4-Azidomethyl-phenyl)-acrylic acid methyl ester

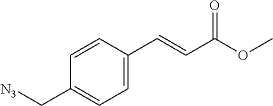

To a solution of trifluoroacetate of 3-(4-Aminomethyl-phenyl)-acrylic acid methyl ester (1.5 g, 5.2 mmol) in methanol (50 mL) were added K₂CO₃ (2.3 g, 11.5 mmol), imadazole sulfonyl azide hydrochloride (1.97 g, 9.4 mmol), CuSO₄.5H₂O (90 mg) and the mixture was stirred at 25° C. over a period of 2 h. The residue obtained upon evaporation of methanol was diluted with ethyl acetate (200 mL), washed with 1.5 N HCl (2×50 mL), water (2×50 mL) and dried over sodium sulphate. The crude product was further purified by column chromatography to obtain 3-(4-Azidomethyl-phenyl)-acrylic acid methyl ester as yellow color solid (0.9 g, 56%).

### Step 7: Preparation of 3-{4-[4-(4-Pyrrolidin-1-ylmethyl-phenyl)-[1,2,3]triazol-1-ylmethyl]-phenyl}-acrylic acid methyl ester

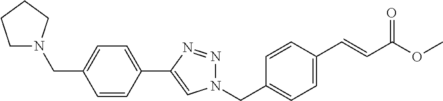

To a solution of 1-(4-Ethynyl-benzyl)-pyrrolidine (0.6 g, 3.2 mmol) in DMF (2 mL) were added Hunig's base (1.6 mL, 9.7 mmol), 3-(4-Azidomethyl-phenyl)-acrylic acid methyl ester (0.7 g, 3.2 mmol), sodium ascorbate (0.3 g, 1.6 mmol) and CuI (0.3 g, 1.6 mmol) and the reaction mixture was stirred at 25° C. over a period of 4 h. The resulting mixture was quenched with ammonia (2 mL), diluted with chloroform (200 mL), washed with water (3×50 mL), dried over sodium sulphate and concentrated. The residue obtained upon evaporation of volatiles was washed with ether to give 3-{4-[4-(4-Pyrrolidin-1-ylmethyl-phenyl)-[1,2,3]triazol-1-ylmethyl]-phenyl}-acrylic acid methyl ester as an off-white solid (0.6 g, 46%).

### Step 8: Preparation of 3-{4-[4-(4-Pyrrolidin-1-ylmethyl-phenyl)-[1,2,3]triazol-1-ylmethyl]-phenyl}-acrylic acid

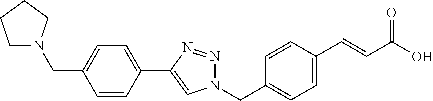

To a suspension of 3-{4-[4-(4-Pyrrolidin-1-ylmethyl-phenyl)-[1,2,3]triazol-1-ylmethyl]-phenyl}-acrylic acid methyl ester in methanol:water (20:3 mL) was added NaOH (0.7 g, 17.9 mmol) at 25° C. and the mixture was stirred at same temperature over a period of 12 h. The methanol was evaporated under reduced pressure, reaction mass was neutralized, and again volatiles were evaporated under reduced pressure to obtain crude mass. The crude mass was diluted with 20% methanol in chloroform and filtered to remove sodium chloride. The residue obtained upon evaporation of volatiles was washed with ether to give 3-{4-[4-(4-Pyrrolidin-1-ylmethyl-phenyl)-[1, 2, 3]triazol-1-ylmethyl]-phenyl}-acrylic acid as a colourless solid (0.58 g, 100%).

### Step 9: Preparation of 3-{4-[4-(4-Pyrrolidin-1-ylmethyl-phenyl)-[1,2,3]triazol-1-ylmethyl]-phenyl}-N-(tetrahydro-pyran-2-yloxy)-acrylamide

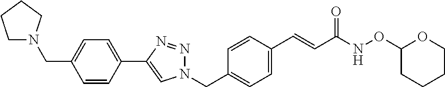

To a suspension 3-{4-[4-(4-Pyrrolidin-1-ylmethyl-phenyl)-[1,2,3]triazol-1-ylmethyl]-phenyl}-acrylic acid (0.5 g, 1.2 mmol) in DMF (3 mL), were added hunig's base (1.1 mL, 6.4 mmol), EDC.HCl (0.7 g, 3.8 mmol), HOBt (0.1 g, 0.64 mmol), O-(Tetrahydro-pyran-2-yl)-hydroxylamine hydrochloride (0.16 g, 1.4 mmol) at 25° C. and the reaction mixture was stirred at same temperature over a period of 2 h. The resulting mixture was diluted with ethyl acetate (500 mL), washed with water (3×100 mL), and dried over sodium sulphate. The reaction mixture was further purified by column chromatography to obtain 3-{4-[4-(4-Pyrrolidin-1-ylmethyl-phenyl)-[1,2,3]triazol-1-ylmethyl]-phenyl}-N-(tetrahydro-pyran-2-yloxy)-acrylamide as an off-white solid (0.2 g, 33%).

### Step 10: Preparation of N-Hydroxy-3-{4-[4-(4-pyrrolidin-l-ylmethyl-phenyl)-[1,2,3]triazol-1-ylmethyl]-phenyl -acrylamide

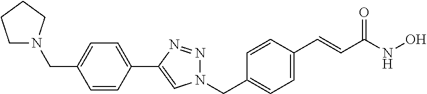

To a solution of 3-{4-[4-(4-Pyrrolidin-1-ylmethyl-phenyl)-[1,2,3]triazol-1-ylmethyl]-phenyl}-N-(tetrahydro-pyran-2-yloxy)-acrylamide (100 mg, 0.19 mmol) in methanol (5 mL) was added 4 N HCl in dioxane (0.02 mL, 0.09 mmol) at 25° C. and the reaction mixture was stirred at same temperature over a period of 60 min. The precipitate formed was separated by filtration and dried under reduced pressure to obtain N-Hydroxy-3-{4-[4-(4-pyrrolidin-1-ylmethyl-phenyl)-[1,2,3]triazol-1-ylmethyl]-phenyl}-acrylamide as an off-white solid (40 mg, 50%).

¹H NMR (300 MHz, DMSO-d₆) δ (ppm): 1.88-2.00 (m, 4H), 3.05-3.16 (m, 2H), 3.32-3.39 (m, 2H), 4.34 (d, J=5.4 Hz, 2H), 5.68 (s, 2H), 6.48 (d, J=15.9 Hz, 1H), 7.33-7.59 (m, 5H), 7.67 (d, J=8.1 Hz, 2H), 7.91 (d, J=7.8 Hz, 2H), 8.71 (s, 1H), 10.91 (bs, 2H).

LCMS (ESI) m/z: 404.1 ([M+H]⁺).

The following compounds were synthesized by using the procedure disclosed above or analogous to the above procedure

| Ex. No | Structure | Analytical data |
|---|---|---|
| 17 | 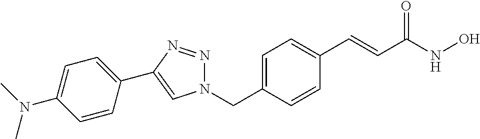 | ¹H NMR (300 MHz, DMSO-d₆) δ (ppm): 2.92 (s, 6H), 5.62 (s, 2H), 6.45 (d, J = 15.9 Hz, 1H), 6.77-6.83 (m, 2H), 7.22-7.75 (m, 7H), 8.41 (s, 1H) LCMS (ESI) m/z: 364.2 ([M + H]⁺). |
| 18 | 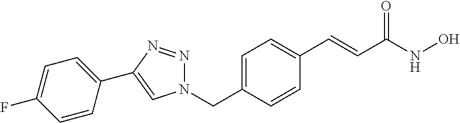 | ¹H NMR (300 MHz, DMSO-d₆) δ (ppm): 5.67 (s, 2H), 6.46 (d, J = 15.6 Hz, 1H), 7.25-7.91 (m, 10H), 8.63 (s, 1H), 10.76 (bs, 1H) LCMS (ESI) m/z: 339.4 ([M + H]⁺). |
| 19 | 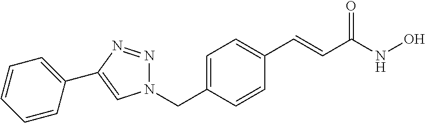 | ¹H NMR (300 MHz, DMSO-d₆) δ (ppm): 5.67 (s, 2H), 6.47 (d, J = 15.6 Hz, 1H), 7.30-7.85 (m, 11H), 8.66 (s, 1H), 10.83 (bs, 1H) LCMS (ESI) m/z: 321.3 ([M + H]⁺). |
| 20 | 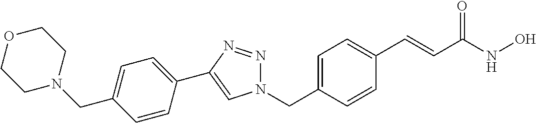 | ¹H NMR (300 MHz, DMSO-d₆) δ (ppm): 2.00 (bs, 4H), 3.45 (d, J = 10.2 Hz, 2H), 3.57 (bs, 4H), 5.66 (s, 2H), 6.45 (d, J = 15.9 Hz, 1H), 7.35- 7.80 (m, 9H), 8.61 (s, 1H), 9.05 (bs, 1H), 10.77 (bs, 1H). LCMS (ESI) m/z: 420.1 ([M + H]⁺). |
| 21 | 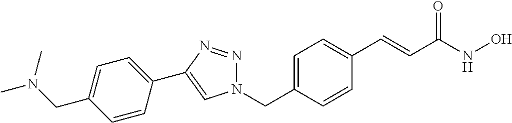 | ¹H NMR (300 MHz, DMSO-d₆) δ (ppm): 2.68 (s, 6H), 4.28 (s, 2H), 5.68 (s, 2H), 5.66 (s, 2H), 6.48 (d, J = 15.6 Hz, 1H), 7.36-7.65 (m, 7H), 7.92 (d, J = 7.8 Hz, 2H) 8.73 (s, 1H), 10.84 (bs, 1H). LCMS (ESI) m/z: 378.1 ([M + H]⁺). |

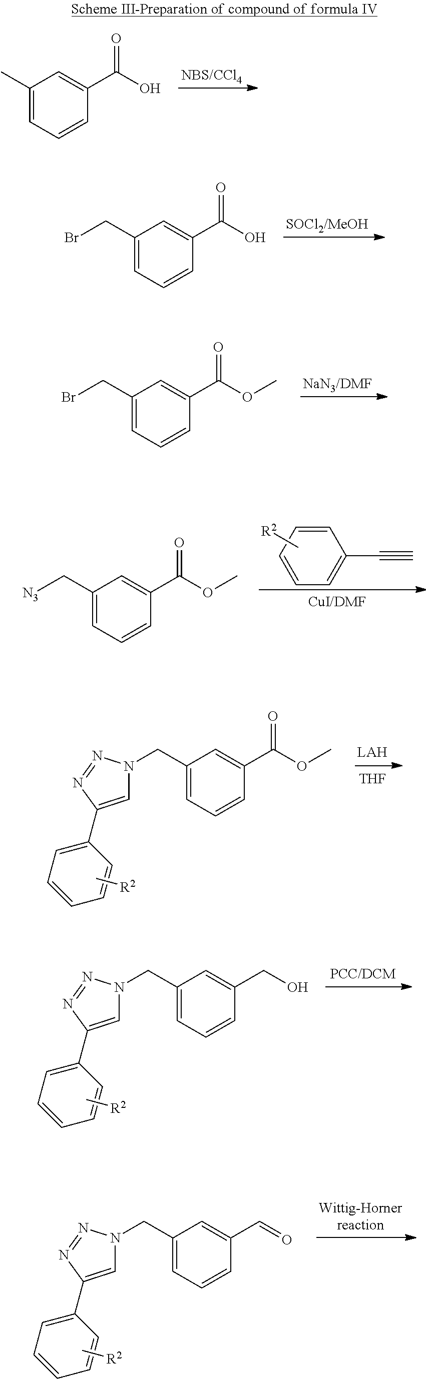

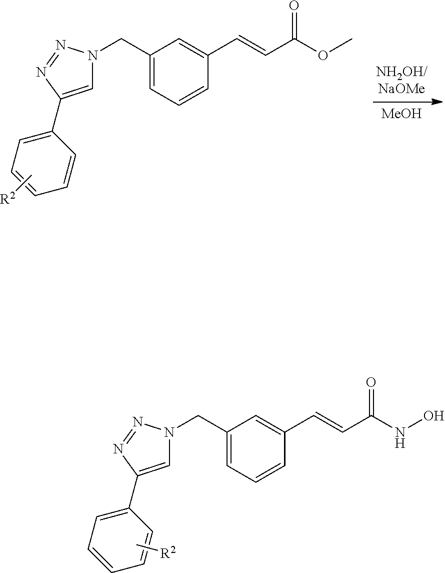

### Example 22

### Synthesis of (E)-N-Hydroxy-3-[3-(4-phenyl-[1, 2, 3]triazol-1-ylmethyl)-phenyl]-acrylamide

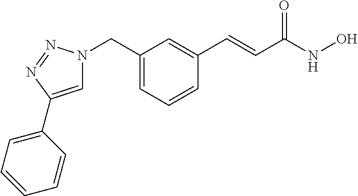

### Step 1: Preparation of 3-Bromomethyl-benzoic acid

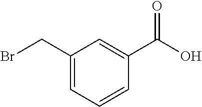

To a suspension of 3-Methyl-benzoic acid (40.0 g, 293 mmol) in carbon tetrachloride (400 mL) were added AIBN (1 g, 0.58 mmol) and N-Bromosuccinamide (52.0 g, 8.07 mmol) at 25° C. The reaction mixture was refluxed over a period of 3 h. The resulting reaction mixture was filtered when it was hot and the filtrate was diluted with ethyl acetate, washed with water and dried over sodium sulphate. The solvent was evaporated under reduced pressure to obtain 3-Bromomethyl-benzoic acid as a colorless solid (54 g, 85%).

### Step 2: Preparation of 3-Bromomethyl-benzoic acid methyl ester

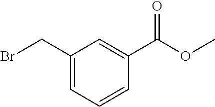

To a solution of 3-Bromomethyl-benzoic acid (1 g, 4.6 mmol) in methanol (20 mL) was added thionyl chloride (1.1 g, 9.3 mmol) drop wise at 25° C. The reaction mixture was refluxed over a period of 1 h and methanol was removed under reduced pressure to obtain crude sticky mass. The crude mass was diluted with ethyl acetate and washed with water and dried over sodium sulphate. Ethyl acetate was evaporated under reduced pressure to obtain 3-Bromomethyl-benzoic acid methyl ester as light yellow oil (1.0 g, 94.0%)

### Step 3: Preparation of 3-Azidomethyl-benzoic acid methyl ester

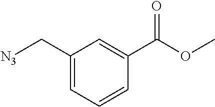

To a solution of 3-Bromomethyl-benzoic acid methyl ester (5.0 g, 21.83 mmol) in DMF (20 mL) was added sodium azide (43.66 mmol) at rt. The reaction mixture was stirred at 80° C. over a period of 3 h. The resulting mixture was diluted with ethyl acetate and washed with water, brine and dried over sodium sulfate. The solvent was evaporated under reduced pressure to obtain 3-Azidomethyl-benzoic acid methyl ester as light yellow oil (3.5 g, 83%).

### Step 4: Preparation of 3-(4-Phenyl-[1,2,3]triazol-1-ylmethyl)-benzoic acid methyl ester

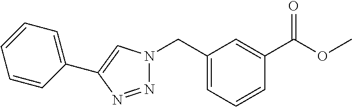

To a solution of 3-Azidomethyl-benzoic acid methyl ester (200 mg, 1.02 mmol) in DMF (5 mL) were added copper iodide (130 mg, 0.68 mmol), sodium ascorbate (70 mg, 0.34 mg), N-ethyl diisopropyl amine (180 mg, 1.37 mmol) and ethynyl-benzene (71 mg, 0.68 mmol) at 25° C. The reaction mixture was stirred at same temperature over a period of 12 h. Then the resulting mixture was quenched with aqueous ammonia and diluted with ethyl acetate. The organic layer washed with water, brine, dried over sodium sulfate and it was evaporated under reduced pressure to obtain crude product. The dude product was further purified by column chromatography to obtain 3-(4-Phenyl-[1,2,3]triazol-1-ylmethyl)-benzoic acid methyl ester as off-white solid (140 mg, 70%).

### Step 5: Preparation [3-(4-Phenyl-[1,2,3]triazol-1-ylmethyl)-phenyl]-methanol

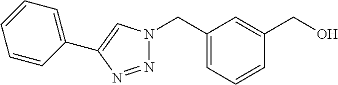

To a suspension of LAH (54 mg, 1.43 mmol) in dry THF (3 mL) was added a solution of 3-(4-Phenyl-[1,2,3]triazol-1-ylmethyl)-benzoic acid methyl ester (140 mg, 0.47 mmol) in dry THF (2 mL) at ice temperature. The reaction mixture was stirred at same temperature over a period of 60 min. The resulting mixture was quenched with saturated ammonium chloride and diluted with ethyl acetate. The ethyl acetate layer was washed with water, brine and dried over sodium sulfate. The solvent was evaporated under reduced pressure and the crude product was purified by column chromatography to obtain [3-(4-Phenyl-[1,2,3]triazol-1-ylmethyl)-phenyl]-methanol as a sticky mass (120 mg, 95%).

### Step 6: Preparation of 3-(4-Phenyl-[1, 2, 3]triazol-1-ylmethyl)-benzaldehyde

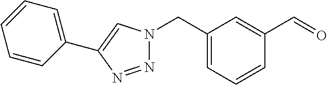

To a solution of [3-(4-Phenyl-[1,2,3]triazol-1-ylmethyl)-phenyl]-methanol (120 mg, 0.45 mmol) in DCM (10 mL) were added sodium acetate (7.4 mg, 0.09 mmol) followed by pyridinium chloro chromate (98 mg, 0.45 mmol) at 25° C. The reaction mixture was stirred at 25° C. over a period of 2 h. The resulting reaction mixture was filtered through celite pad and the filtrate was diluted with dichloromethane, washed with water and dried over sodium sulphate. Then solvent was evaporated under reduced pressure and the crude product was purified through column chromatography to give 3-(4-Phenyl-[1,2,3]triazol-1-ylmethyl)-benzaldehyde as a sticky mass (80 mg, 66%).

### Step 7: (E)-3-[3-(4-Phenyl-[1,2,3 ]triazol-1-ylmethyl)-phenyl]-acrylic acid methyl ester

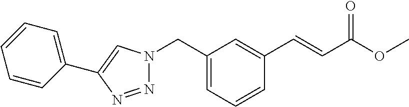

To a solution of trimethyl phosphino acetate (40 mg, 0.60 mmol) in dry DMF (3 mL) was added potassium tert-butoxide (51 mg, 0.45 mmol) at 0° C. and the mixture was stirred at same temperature over a period of 15 min. To the above mixture, a solution of 3-(4-Phenyl-[1,2,3]triazol-1-ylmethyl)-benzaldehyde (80 mg, 0.30 mmol) in DMF (1 mL) was added drop wise at ice temperature. The reaction mixture was stirred at ice temperature over a period of 45 min. The resulting reaction mixture was diluted with ethyl acetate, washed with water, brine, dried over sodium sulfate and it was evaporated under reduced pressure to obtain crude product. The crude product was purified by column chromatography to obtain (E)-3-[3-(4-Phenyl-[1,2,3]triazol-1-ylmethyl)-phenyl]acrylic acid methyl ester as off-white solid (80 mg, 82%).

### Step 8: Preparation of (E)-N-Hydroxy-3-[3-(4-phenyl-[1,2,3]triazol-1-ylmethyl)-phenyl]-acrylamide

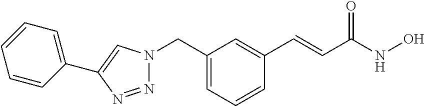

To a suspension of hydroxyl amine hydrochloride (152 mg, 2.19 mmol) in methanol (5.0 mL) was added sodium methoxide (153 mg, 2.85 mmol) at ice temperature and the suspension was stirred at ice temperature over a period of 30 min. To the above suspension (E)-3-[3-(4-Phenyl-[1,2,3]triazol-1-ylmethyl)-phenyl]-acrylic acid methyl ester in methanol:DCM (4:1) was added drop wise at −20° C. The reaction temperature was allowed to reach 25° C. and stirred at same temperature over a period of 3 h. The resulting reaction mixture was acidified with hydrochloric acid and the solvents were removed under reduced pressure to obtain crude solid. The crude solid was further purified by column chromatography to give (E)-N-Hydroxy-3-[3-(4-phenyl-[1,2,3]triazol-1-ylmethyl)-phenyl]-acrylamide as a pinkish solid (25 mg, 35.0%).

¹H NMR (300 MHz, DMSO-d₆) δ (ppm): 5.67 (s, 2H), 6.47 (d, J=15.9 Hz, 1H), 7.58-7.30 (m, 8H), 7.84 (d, J=7.5 Hz, 2H), 8.66 (s, 1H), 10.81 (bs, 1H).

LCMS (ESI) m/z: 321.3 ([M+H]⁺).

The following compounds were synthesized by using the procedure disclosed above or analogous to the above procedure

| Ex. No | Structure | Analytical data |
|---|---|---|
| 23 | 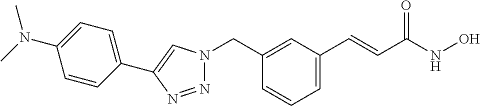 | ¹H NMR (300 MHz, DMSO-d₆) δ (ppm): 2.92 (s, 6H), 5.62 (s, 2H), 6.45 (d, J = 15.6 Hz, 1H), 6.75 (d, J = 7.2 Hz, 2H), 7.34-7.65 (m, 7H), 8.43 (s, 1H). LCMS (ESI) m/z: 364.3 ([M + H]⁺). |
| 24 | 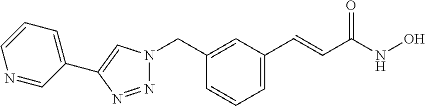 | LCMS (ESI) m/z: 322.2 ([M + H]⁺). |
| 25 | 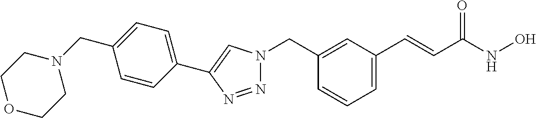 | ¹H NMR (300 MHz, DMSO-d₆) δ (ppm): 2.72-3.24 (m, 4H), 3.52- 3.94 (m, 4H), 4.54 (s, 2H), 5.69 (s, 2H), 6.48 (d, J = 15.9 Hz, 1H), 7.26-7.79 (m, 7H), 7.93 (d, J = 7.8 Hz, 2H), 8.74 (s, 1H). LCMS (ESI) m/z: 420.2 ([M + H]⁺). |
| 26 | 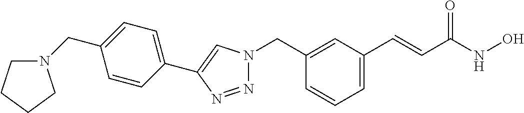 | ¹H NMR (300 MHz, DMSO-d₆) δ (ppm): 1.87 (s, 2H), 2.00 (s, 2H), 3.04 (s, 2H), 3.32 (s, 2H), 4.33 (s, 2H), 5.64 (s, 2H), 6.48 (d, J = 15.9 Hz, 1H), 7.33-7.67 (m, 7H), 7.92 (d, J = 7.8 Hz, 2H), 8.73 (s, 1H). LCMS (ESI) m/z: 404.3 ([M + H]⁺). |
| 27 | 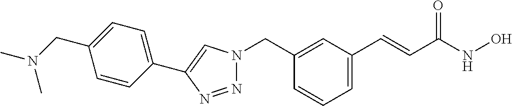 | ¹H NMR (300 MHz, DMSO-d₆) δ (ppm): 2.69 (s, 3H), 2.70 (s, 3H), 4.28 (s, 2H), 5.69 (s, 2H), 6.45 (d, J = 15.6 Hz, 1H), 7.33-7.62 (m, 7H), 7.93 (d, J = 7.2 Hz, 2H), 8.73 (s, 1H), 10.45 (bs, 1H), 10.84 (s, 1H). LCMS (ESI) m/z: 378.3 ([M + H]⁺). |

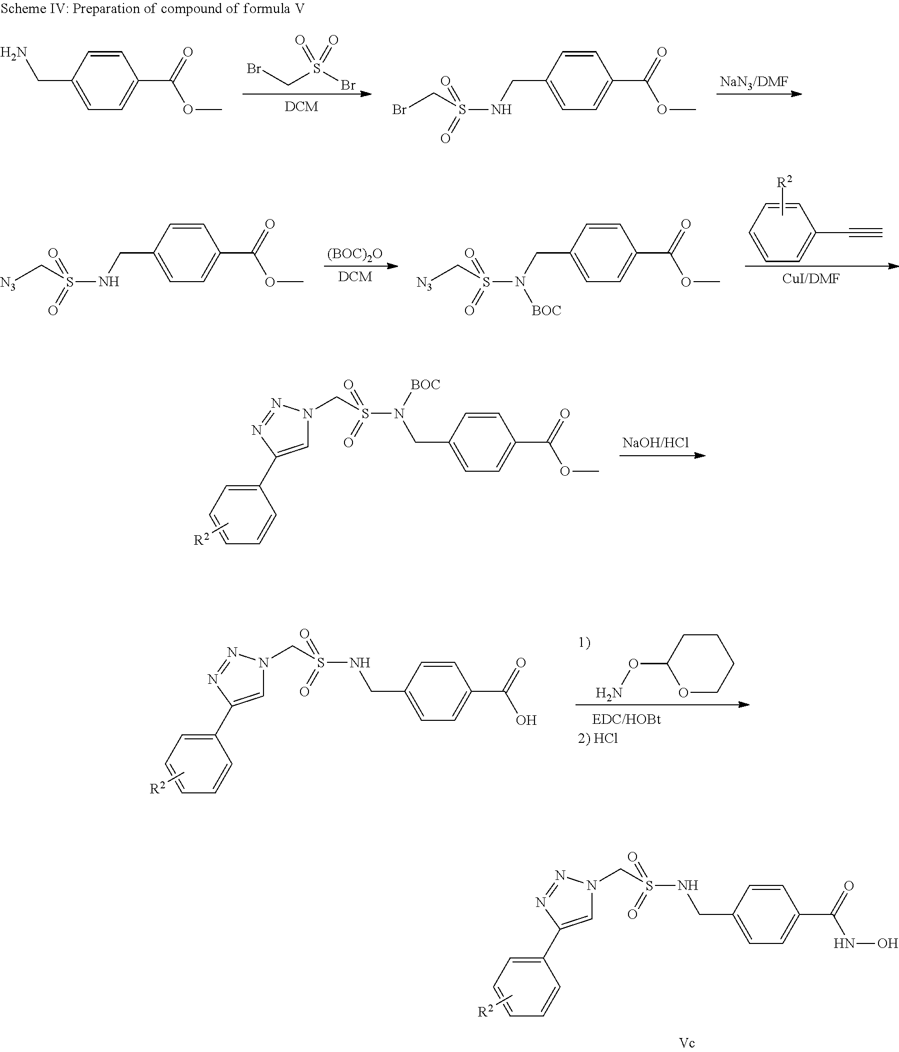

### Example 28

### Preparation of N-Hydroxy-4-[(4-phenyl-[1,2,3]triazol-1-ylmethanesulfonyl amino)-methyl]-benzamide (1)

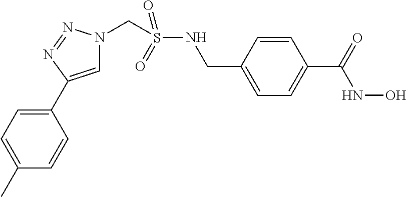

### Step 1: Preparation of 4-(Bromomethanesulfonylamino-ethyl)-benzoic acid methyl ester

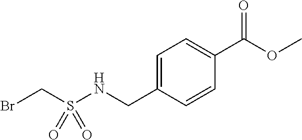

To a solution of 4-Aminomethyl-benzoic acid methyl ester (15.0 g, 0.10 mol) in dichloromethane (150 mL) was added triethylamine (45.2 mL, 0.29 mol) and bromo-methanesulfonyl bromide (35.9 g, 0.14 mol) at ice temperature. The reaction mixture was stirred at 25° C. over a period of 3 h. The resulting reaction mixture was diluted with dichloromethane (500 mL), washed with water, brine and dried over sodium sulfate. The solvent was evaporated under reduced pressure and the residue obtained was further purified by column chromatography to give 4-(Bromomethanesulfonylamino-ethyl)-benzoic acid methyl ester as pale yellow solid (13 g, 39%).

### Step 2: Preparation of 4-(Azidomethanesulfonylamino-methyl)-benzoic acid methyl ester

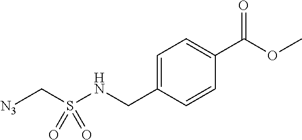

To a solution of 4-(Bromomethanesulfonylamino-ethyl)-benzoic acid methyl ester (10 g, 31.0 mmol) in DMF (100 mL) was added sodium azide (4.0 g, 62.0 mmol) at 25° C. The reaction mixture was stirred at 80° C. over a period of 2 h. The resulting mixture was diluted with ethyl acetate (1000 mL) and washed with water, brine and dried over sodium sulfate. The solvent was evaporated under reduced pressure to obtain 4-(Azidomethanesulfonylamino-methyl)-benzoic acid methyl ester as pale yellow solid (7.0 g, 79.0%).

### Step 3: Preparation of 4-[(Azidomethanesulfonyl-tert-butoxycarbonylamino)-methyl]-benzoic acid methyl ester

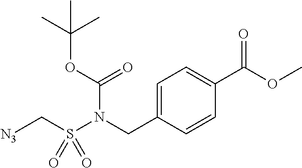

To a solution of 4-(Azidomethanesulfonylamino-methyl)-benzoic acid methyl ester (0.2 g, 0.6 mmol) in DCM (10 mL) was added carbonic acid di-tert-butyl ester (0 3 mL, 1 mmol) followed by catalytic amount of DMAP at 0° C. The reaction mixture was stirred at 25° C. over a period of 12 h. The resulting mixture was diluted with DCM (30 mL), washed with water and dried over sodium sulfate. The solvent was evaporated under reduced pressure to obtain 4-[(Azidomethanesulfonyl-tert-butoxycarbonylamino)-methyl]-benzoic acid methyl ester as an oil (0.23 g, 85.0%).

### Step 4: Preparation of 4-{[tert-Butoxycarbonyl-(4-p-tolyl-[1,2,3]triazol-1-ylmethanesulfonyl)-amino]-methyl}-benzoic acid methyl ester

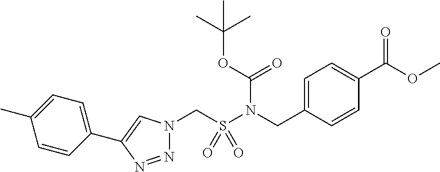

To a solution of 4-[(Azidomethanesulfonyl-tert-butoxycarbonylamino)-methyl]-benzoic acid methyl ester (2.0 g, 5.1 mmol) in DMF (10 mL) were added 1-Ethynyl-4-methyl-benzene (600 mg, 5.1 mmol), copper iodide (490 mg, 2.5 mmol), sodium ascorbate (510 mg, 2.5 mmol) and N-ethyl diisopropyl amine (2.6 mL, 15.5 mmol) at 25° C. The reaction mixture was stirred at same temperature over a period of 12 h. Then the resulting mixture was quenched with aqueous ammonia and diluted with ethyl acetate. The organic layer washed with water, brine and dried over sodium sulfate. The residue obtained upon evaporation of the volatiles was purified by column chromatography to give 4-{[tert-Butoxycarbonyl-(4-p-tolyl-[1,2,3]triazol-1-ylmethanesulfonyl)-amino]-methyl}-benzoic acid methyl ester as a yellow solid (600 mg, 23%).

### Step 5: Preparation of 4-[(4-p-Tolyl-[1,2,3]triazol-1-ylmethanesulfonylamino)-methyl]-benzoic acid

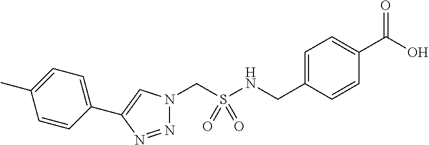

To a solution of 4-[tert-Butoxycarbonyl-(4- p-tolyl-[1,2,3]triazol-1-ylmethanesulfonyl)-amino]-methyl}-benzoic acid methyl ester (600 mg, 1.1 mmol) in methanol (15.0 mL) was added NaOH in water (4.0 mL) at25° C. The reaction mixture was stirred at same temperature over a period of 12 h. The solvent was evaporated under reduced pressure and the crude product was acidified with 1.5 N HCl to obtain the precipitate in aqueous solution. The precipitate was filtered and dried to obtain 4-[(4-p-Tolyl-[1,2,3]triazol-1-ylmethanesulfonylamino)-methyl]-benzoic acid as off-white solid (400 mg, 86.0%).

### Step 6: Preparation of N-Hydroxy-4-[(4- p-Tolyl-1,2,3]triazol-1-ylmethanesulfonylamino)-methyl]-benzamide

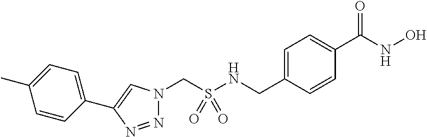

To a solution of 4-[(4-p-Tolyl-[1,2,3]-triazol-1-ylmethanesulfonylamino)-methyl]-benzoic acid (400 mg, 1.03 mmol) in DMF (3.0 mL) were added N-Ethyl diisopropyl amine (0.88 mL, 5.1 mmol), EDC.HCl (590 mg, 3.1 mmol), HOBt (70 mg, 0.51 mmol), and O-(Tetrahydro-pyran-2-yl)-hydroxylamine hydrochloride (130 mg, 1.13 mmol) in DMF at ice temperature under nitrogen atmosphere. The reaction mixture was stirred at 25° C. over a period of 4 h. Then the resultant reaction mixture was triturated in to the diethyl ether and the precipitated sticky mass was washed with water to obtain (360 mg, 72%) of the 4-[(4-p-Tolyl-[1,2,3]triazol-1-ylmethanesulfonylamino)-methyl]-N-(tetrahydro-pyran-2-yloxy)-benzamide as a colorless solid.

100 mg (2.0 mmol) of the above solid was suspended in methanol (5.0 mL) and added cat. amount of dry HCl in dioxane and stirred for 30 min. Then the solvent was evaporated under reduced pressure to obtain N-Hydroxy-4-[(4-p-Tolyl-1,2,3]triazol-1-ylmethanesulfonylamino)-methyl]-benzamide as a colorless solid (40 mg, 50%).

¹H NMR (300 MHz, DMSO-d₆) δ (ppm): 2.34 (s, 3H), 4.24 (d, J=6.0 Hz, 2H), 6.00 (s, 2H), 7.27 (d, J=7.8 Hz, 2H), 7.39 (d, J=8.4 Hz, 2H), 7.72 (d, J=8.1 Hz, 2H), 7.79 (d, J=8.1 Hz, 2H), 8.30 (bs, 1H), 8.55 (s, 1H), 9.03 (s, 1H), 11.21(s, 1H).

LCMS (EST) m/z: 402.1 ([M+H]⁺).

The following compounds were synthesized by using the procedure disclosed above or analogous to the above procedure

| Ex. No | Structure | Analytical data |
|---|---|---|
| 29 | 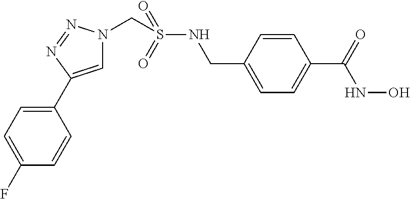 | ¹H NMR (300 MHz, DMSO-d₆) δ (ppm): 4.24 (d, J = 6.0 Hz, 2H), 6.02 (s, 2H), 7.28-7.41 (m, 4H), 7.73 (d, J = 8.1 Hz, 2H), 7.93-7.98 (m, 2H), 8.30 (t, J = 6.0 Hz, 1H), 8.63 (s, 1H), 11.20 (s, 1H). LCMS (ESI) m/z: 406.1 ([M + H]⁺). |
| 30 | 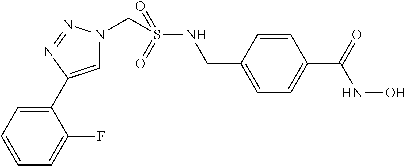 | ¹H NMR (300 MHz, DMSO-d₆) δ (ppm): 4.23 (s, 2H), 6.07 (s, 2H), 7.33-7.48 (m, 6H), 7.72 (d, J = 8.1 Hz, 2H), 8.15-8.20 (m, 1H), 8.48 (s, 1H), 9.02 (brs, 1H), 11.23 (s, 1H). LCMS (ESI) m/z: 406.1 ([M + H]⁺). |
| 31 | 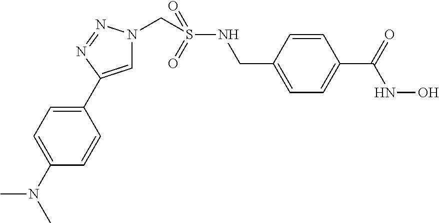 | ¹H NMR (300 MHz, DMSO-d₆) δ (ppm): 2.98 (s, 6H), 4.23 (d, J = 5.7 Hz, 2H), 5.97 (s, 2H), 6.92- 6.97 (m, 2H), 7.39 (d, J = 8.1 Hz, 2H), 7.71-7.79 (m, 4H), 8.29 (t, J = 6.0 Hz, 1H), 8.44 (s, 1H), 11.20 (bs, 1H). LCMS (ESI) m/z: 430.8 ([M + H]⁺). |
| 32 | 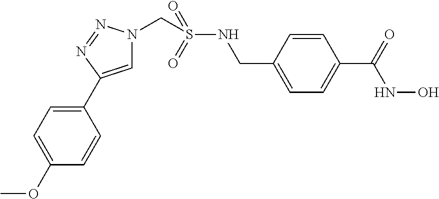 | ¹H NMR (300 MHz, DMSO-d₆) δ (ppm): 3.79 (s, 3H), 4.23 (s, 2H), 5.99 (s, 2H), 7.03 (d, J = 8.7 Hz, 2H), 7.40 (d, J = 8.1 Hz, 2H), 7.73 (d, J = 7.8 Hz, 2H), 7.83 (d, J = 8.7 Hz, 2H), 8.51 (s, 1H). LCMS (ESI) m/z: 418.1 ([M + H]⁺). |
| 33 | 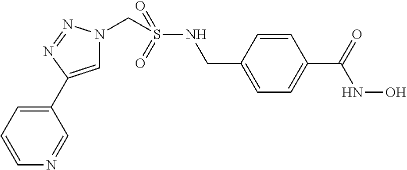 | ¹H NMR (300 MHz, DMSO-d₆) δ (ppm): 4.25 (s, 2H), 6.06 (s, 2H), 7.10-7.23 (m, 1H), 7.40 (d, J = 7.8 Hz, 2H), 7.49-7.53 (m, 1H), 7.73 (d, J = 7.8 Hz, 2H), 8.29 (d, J = 7.8 Hz, 2H), 8.57 (d, J = 4.5 Hz, 2H), 8.80 (s, 1H), 9.02 (brs, 1H), 9.13 (s, 1H), 11.23 (s, 1H). LCMS (ESI) m/z: 389.1 ([M + H]⁺). |
| 34 | 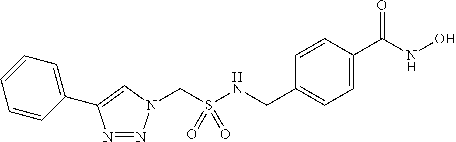 | ¹H NMR (300 MHz, DMSO-d₆) δ (ppm): 4.24 (s, 2H), 6.01 (s, 2H), 5.68 (s, 2H), 7.36-7.60 (m, 5H), 7.73 (d, J = 8.4 Hz, 2H), 7.90 (d, J = 7.2 Hz, 2H), 8.62 (s, 1H). LCMS (ESI) m/z: 387.9 ([M + H]⁺). |

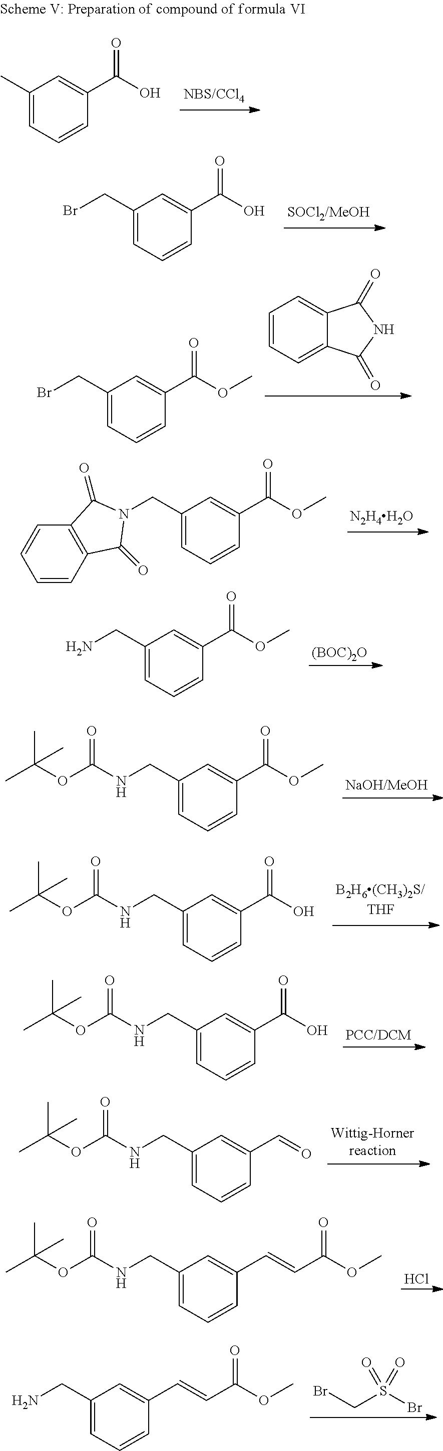

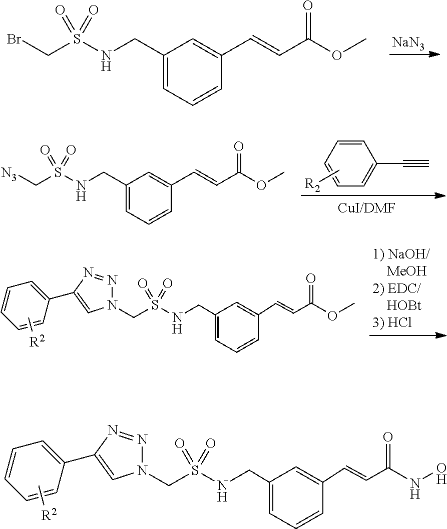

### Example 35

### Synthesis of N-Hydroxy-3-{3-[(4-phenyl-[1,2,3]triazol-1-ylmethanesulfonyl amino)-methyl]-phenyl}-acrylamide

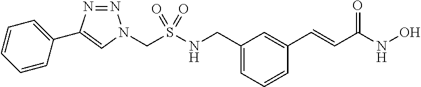

### Step 1: Preparation of 3-(1, 3-Dioxo-1,3-dihydro-isoindol-2-ylmethyl)-benzoic acid methyl ester

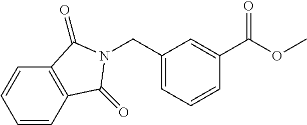

To a solution of 3-Bromomethyl-benzoic acid methyl ester (25.6 g, 111.7 mmol) in DMF was added potassium phthalimide at rt and the reaction mixture stirred 85° C. over a period of 12 h. The resulting mixture was diluted with ethyl acetate (1.5 Lit), washed with water (5×300 mL), brine (300 mL) and dried over sodium sulphate. Ethyl acetate was evaporated under reduced pressure to obtain 3-(1,3-Dioxo-1,3-dihydro-isoindol-2-ylmethyl)-benzoic acid methyl ester colourless solid (30.0 g, 95.5%)

### Step 2: Preparation of 3-Aminomethyl-benzoic acid methyl ester hydrochloride

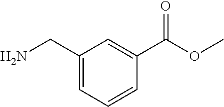

To a suspension of 3-(1,3-Dioxo-1,3-dihydro-isoindol-2-ylmethyl)-benzoic acid methyl ester (0.5 g, 1.70 mmol) in methanol (15 mL) was added hydrazine mono hydrate (0.08 mL) at 25° C. and the reaction mixture was refluxed over a period of 4 h and it was allowed to reach 25° C. The white suspension formed was filtered and the filtrate was concentrated. The filtrate obtained was diluted with water (20 mL) and acidified with 1.5 N HCl and water was evaporated under reduced pressure to obtain 0.5 g) of the 3-Aminomethyl-benzoic acid methyl ester hydrochloride as a crude solid. The crude product obtained was used for next step without further purification.

### Step 3: Preparation of 3-(tert-Butoxycarbonylamino-methyl)-benzoic acid methyl ester

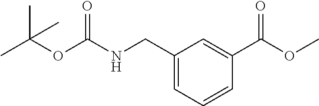

The crude product obtained in the previous step was diluted with water, basified (P^{H}=8.0) with aq. Sodium bicarbonate. To this mixture a solution of Di-tert-butyl dicarbonate (0.79 mL, 3 40 mmol) in ethyl acetate (10 mL) was added drop wise over a period of 10 min and it was stirred over a period of 60 min. The reaction mixture was diluted with ethyl acetate (100 mL), washed water, brine and dried over sodium sulphate. Solvent was removed under reduced pressure to give 3-(tert-Butoxycarbonylamino-methyl)-benzoic acid methyl ester as a colourless solid (0.6 g, 92%).

### Step 4: Preparation of 3-(tert-Butoxycarbonylamino-methyl)-benzoic acid

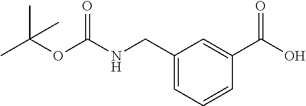

To a solution of 3-(tert-Butoxycarbonylamino-methyl)-benzoic acid methyl ester (0.6 g, 2.20 mmol) in THF (4 mL) was added lithium hydroxide (0.3 g, 9.0 mmol) in water (2 mL) at 25° C. The reaction mixture was stirred at 50° C. over a period of 4 h. THF was removed under reduced pressure, acidified with acetic acid and the product was extracted with ethyl acetate, dried over sodium sulphate. The crude product was purified by column chromatography to give 3-(tert-Butoxycarbonylamino-methyl)-benzoic acid as a colourless solid (250 mg, 44%)

### Step 5: Preparation of (3-Hydroxymethyl-benzyl)-carbamic acid tert-butyl ester

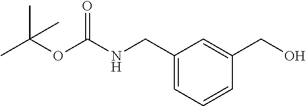

To a solution of 3-(tert-Butoxycarbonylamino-methyl)-benzoic acid (250 mg, 0.99 mmol) in dry THF (4.0 mL) was added borane dimethyl sulfide (0.37 mL, 3.9 mmol) drop wise at ice temperature and was stirred at 25° C. over a period of 2 h. The resulting mixture was quenched with sat. NH₄Cl and solvent was evaporated under reduced pressure. Residue obtained was diluted with ethyl acetate (100 mL) and washed with water and dried over sodium sulphate. The solvent was evaporated under reduced pressure to give (3-Hydroxymethyl-benzyl)-carbamic acid tert-butyl ester as yellow oil (200 mg, 86%).

### Step 6: Preparation of (3-Formyl-benzyl)-carbamic acid tert-butyl ester

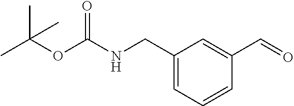

To a suspension of PCC (360 mg, 1.6 mmol) and sodium acetate (26 mg, 0.32 mmol) in dichloremethane (5.0 mL) was added (3-Hydroxymethyl-benzyl)-carbamic acid tert-butyl ester (200 mg, 0.84 mmol) in dichloromethane (5.0 mL) and the reaction mixture was stirred at 25° C. over a period of 6 h. The resulting mixture was diluted with ethyl acetate (50 mL), filtered through celite pad, the filtrate was washed with water, brine and dried over sodium sulphate. The solvent was evaporated under reduced pressure to give (3-Formyl-benzyl)-carbamic acid tert-butyl ester as brownish oil (180 mg, 94%).

### Step 7: Preparation of 3-[3-(tert-Butoxycarbonylamino-methyl)-phenyl]-acrylic acid methyl ester

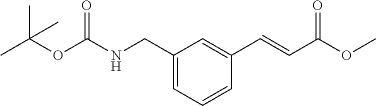

To a suspension of potassium t-butoxide (120 mg, 1.14 mmol) in dry THF (5.0 mL) was added trimethyl phosphiono acetate (24 mL, 1.50 mmol) at ice temperature and was stirred at same temperature over a period of 20 min. To the above suspension a solution of (3-Formyl-benzyl)-carbamic acid tert-butyl ester (180 mg, 0.76 mmol) in THF (5.0 mL) was added drop wise at ice temperature and it was stirred at same temperature over a period of 60 min. The reaction mixture was quenched with ice cold water, diluted with ethyl acetate (100 mL). The ethyl acetate layer was washed with water, dried and concentrated to give 3-[3-(tert-Butoxycarbonylamino-methyl)-phenyl]-acrylic acid methyl ester as a yellow oil (180 mg, 81%).

### Step 8 : Preparation of 3-(3-Aminomethyl-phenyl)-acrylic acid methyl ester hydrochloride

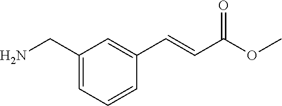

To a solution of 3-[3-(tert-Butoxycarbonylamino-methyl)-phenyl]-acrylic acid methyl ester (90 mg, 0.3 mmol) in methanol (2.0 mL) was added 4M hydrochloric acid in dioxane (0.60 mL, 3.0 mmol) at ice temperature. The mixture was stirred at 25° C. over a period of 6 h. Solvent was evaporated under reduced pressure and the residue obtained was washed with ether to give 3-(3-Aminomethyl-phenyl)-acrylic acid methyl ester hydrochloride as a yellow solid (50 mg, 84%).

### Step 9: Preparation of 3-[3-(Bromomethanesulfonylamino-methyl)-phenyl]-acrylic acid methyl ester

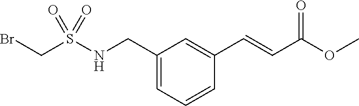

To a suspension of 3-(3-Aminomethyl-phenyl)-acrylic acid methyl ester hydrochloride (0.7 g, 3.6 mmol) in dichloromethatne (20.0 mL) were added triethyl amine (1.4 mL, 10.9 mmol), bromo-methanesulfonyl bromide (4.3 g, 18.3 mmol) at ice temperature. The reaction mixture was stirred at 25° C. over a period 12 h. The reaction mixture was diluted with dichloromethatne (150 mL), washed with water (3×50 mL), dried over sodium sulphate and concentrated. The crude product was further purified by column chromatography to give 3-[3-(Bromomethanesulfonylamino-methyl)-phenyl]-acrylic acid methyl ester as a yellow colour solid 0.5 g (39%)

### Step 10: Preparation of 3-[3-(Azidomethanesulfonylamino-methyl)-phenyl]-acrylic acid methyl ester

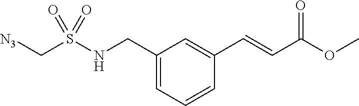

To a solution of 3-[3-(Bromomethanesulfonylamino-methyl)-phenyl]-acrylic acid methyl ester (0.5 g, 1.4 mmol) in DMF (3 mL) was added NaN3 (0.18 g, 2.8 mmol) and the reaction mixture was stirred at 80° C. over a period of 4 h. The reaction mixture was diluted with ethyl acetate (100 mL) and the organic layer was washed with water, brine and dried over sodium sulphate. The solvent was evaporated under reduced pressure to give 3-[3-(Azidomethanesulfonylamino-methyl)-phenyl]-acrylic acid methyl ester as an orange oil (0.4 g, 90%).

### Step 11: 3-{3-[(4-Phenyl-[1,2,3]triazol-1-ylmethanesulfonylamino)-methyl]-phenyl}-acrylic acid methyl ester

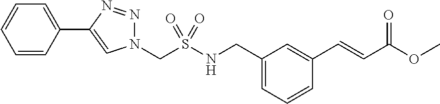

To a solution of 3-[3-(Azidomethanesulfonylamino-methyl)-phenyl]-acrylic acid methyl ester (200 mg, 0.64 mmol) in DMF (3 mL) were added sequentially hunig's base (0.33 mL, 1.9 mmol), ethynyl-benzene (0.08 mL, 0.77 mmol), CuI (0.32 mmol), sodium ascarbate (0.32 mmol) and the mixture was stirred at 25° C. over a period of 5. h. The resulting mixture was quenched with ammonium hydroxide (1 mL), diluted with ethyl acetate (100 mL), washed with water (2×50 mL), dried over sodium sulphate and concentrated. The crude product was further purified by column chromatography to give 3-{3-[(4-Phenyl-[1,2,3]triazol-1-ylmethanesulfonylamino)-methyl]-phenyl}-acrylic acid methyl ester as a off-white solid (120 mg, 46%)

### Step 12: Preparation of 3-{3-[(4-Phenyl-[1,2,3]triazol-1-ylmethanesulfonylamino)-methyl]-phenyl}-acrylic acid

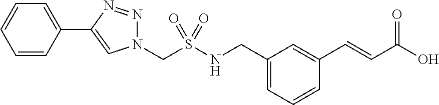

To a suspension of 3-{3-[(4-Phenyl-[1,2,3]triazol-1-ylmethanesulfonylamino)-methyl]-phenyl}-acrylic acid methyl ester (120 mg, 0.29 mmol) in methanol (5 mL) was added NaOH (130 mg, 3.4 mmol) in water (2 mL) and the reaction mixture was stirred at 25° C. over a period of 12 h. The resulting mixture was acidified (P^{H}=2.0) and solid obtained was isolated by filtration to give 3-{3-[(4-Phenyl-[1,2,3]triazol-1-ylmethanesulfonylamino)-methyl]-phenyl}-acrylic acid as an off-white solid (100 mg, 86%).

### Step 13: Preparation of 3-{3-[(4-Phenyl-[1,2,3]triazol-1-ylmethanesulfonylamino)-methyl]-phenyl}-N-(tetrahydro-pyran-2-yloxy)-acrylamide

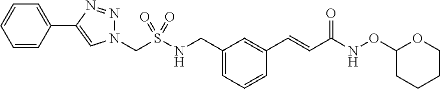

To a solution of 3-{3-[(4-Phenyl-[1,2,3]triazol-1-ylmethanesulfonylamino)-methyl]-phenyl}-acrylic acid (100 mg, 0.25 mmol) in DMF (2 mL) were added sequentially Hunig's base (0.21 mL, 1.2 mmol), EDC.HCl (140 mg, 0.75 mmol), HOBt (19 mg, 0.12 mmol) and O-(Tetrahydro-pyran-2-yl)-hydroxylamine hydrochloride (30 mg, 0.27 mmol) at ice temperature. The reaction mixture was stirred at 25° C. over a period of 2h. The resulting mixture was added triturated in to diethyl ether (50 mL) to obtain sticky product. The sticky mass was washed with water to obtain precipitate and it was separated through filtration to give 3-{3-[(4-Phenyl-υ1,2,3]triazol-1-ylmethanesulfonylamino)-methyl]-phenyl}-N-(tetrahydro-pyran-2-yloxy)-acrylamide as an off-white solid (100 mg, 83%)

### Step 14: Preparation of N-Hydroxy-3-{3-[(4-phenyl-[1,2,3]triazol-1-ylmethanesulfonylamino)-methyl]-phenyl}-acrylamide

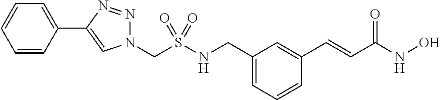

To a suspension of 3-{3-[(4-Phenyl-[1,2,3]triazol-1-ylmethanesulfonylamino)-methyl]-phenyl}-N-(tetrahydro-pyran-2-yloxy)-acrylamide (100 mg, 0.19 mmol) in methanol (5 mL) was added 4M hydrochloric acid in dioxane (0.02 mL, 0.09 mmol) at 25° C. The reaction mixture was stirred at 25° C. over a period of 30 min. The solvent was evaporated under reduced pressure to give N-Hydroxy-3-{3-[(4-phenyl-[1,2,3]triazol-1-ylmethanesulfonylamino)-methyl]-phenyl}-acrylamide as an off-white solid (80 mg, 96%)

¹H NMR (300 MHz, DMSO-d₆) δ (ppm): 4.23 (d, J=6.0 Hz, 2H), 6.01 (s, 2H), 6.48 (d, J=15.6 Hz, 2H), 7.35-7.63 (m, 8H), 7.91 (d, J=7.2 Hz, 2H), 8.29 (t, J=6.0 Hz, 1H), 8.61 (bs, 1H), 10.90 (bs, 1H).

LCMS (ESI) m/z: 414.7 ([M+H]⁺).

The following compound was synthesized by using the procedure disclosed above or analogous to the above procedure

| Ex. No | Structure | Analytical data |
|---|---|---|
| 36 | 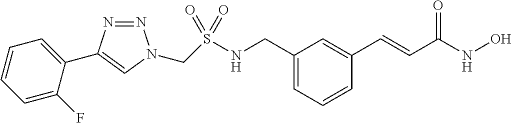 | ¹H NMR (300 MHz, DMSO- d₆) δ (ppm): 4.22 (d, J = 5.7 Hz, 2H), 6.07 (s, 2H), 6.47 (d, J = 15.9 Hz, 2H), 7.35-7.52 (m, 8H), 8.17 (t, J = 7.8 Hz, 1H), 8.28 (t, J = 6.3 Hz, 1H), 8.46 (s, 1H), 10.90 (bs, 1H). LCMS (ESI) m/z: 432.1 ([M + H]⁺). |

### Example 37

### Cell Proliferation Assay

Anticancer activity of the compounds has been tested in NCI-H460 (ATCC NO #HTB-177 Large cell lung cancer), HT-29 (ATCC NO #HTB-38 Colon adenocarcinoma), and A549 (ATCC NO #CCL-185 Lung carcinoma), PC-3 (ATCC NO CRL-1435 prostate adenocarcinoma) and PA-1 (ATCC NO #CRL-1572 Ovarian teratocarcinoma) cell lines by using MTT assay. Cells were maintained in RPMI 1640 with 10% FBS (Fatal Bovine Serum), Penicillin (50 μg/mL), and Streptomycin (100 μg/mL). Cells were seeded in a 96-well cell culture plates at a concentration of 10,000 cells per well and incubated at 37° C. in CO₂ incubator. Separate plate with these cell lines is also inoculated to determine cell viability before the addition of the compounds (T₀)

Compound Addition

Twenty-four hours later cells were treated with different concentrations (100, 10, 1, 0.1, and 0.01 μM) of compound dissolved in DMSO and incubated for 48 hours. For To measurement, 24 hours after seeding the cells, 20 μL of 3-(4.5-dimethyl-2- thiazolyl)-2,5-diphenyl-2H-tetrazolium (MTT) solution per well was added to the T₀ plate and incubated for 3 hours at 37° C. in a CO₂ incubator.

Measurement of Cell Proliferation

The plate containing cells and test compounds was treated similarly after 48 hours of incubation. After 3 hours of MTT addition, well contents were aspirated carefully followed by addition of 100 μL DMSO per well. Plates were agitated to ensure dissolution of the formazan crystals in DMSO and absorbance was read at 570 nm (A570). From the optical densities the percentage growths were calculated using the following formulae: if T is greater than or equal to T0, percentage growth=100×[(T−T0)/(C−T0)] and if T is less than T0, percentage growth=100×[(T−T0)/T0)], where T is optical density of test, C is the optical density of control, and T0 is the optical density at time zero. From the percentage growths a dose response curve was generated and GI50 values were interpolated from the growth curves.

### HDAC Activity Screening

To study the human HDAC inhibition the in-house 96 well plate assay have been established using fluorescent substrate (Boc-Lys (Ac)-AMC Substrate). Hela cells nuclear extract have been used as enzyme source.

Assay was performed in 96-well black microplate and total volume of the assay was 100 μL. Hela nuclear extract was diluted HDAC assay buffer (final concentration of 3.0 μg/mL). Enzyme mixture was made of 10 μL of diluted enzyme and 30 μL of HDAC buffer. 40 μl of enzyme mixture followed by 10 μL of test compound (final concentration from 0.01 to 10 μM) or vehicle (control) was added to each well. The plate was pre-incubated at 37° C. for 10 minutes. The HDAC reaction was started by adding 50 μl of HDAC substrate Boc-Lys (Ac)-AMC (Anaspec, Inc Fremont, Calif., USA). The plate was incubated at 37° C. for 45 minutes. The reaction was stopped by adding 50 μL of Trypsin stop solution and plate further incubated at 37° C. for 15 minutes. Measuring the fluorescence at excitation wavelength of 360 nm and emission wavelength of 460 nm monitored the release of AMC. Buffer alone and substrate alone served as blank. For selected compounds, IC₅₀ (50% HDAC inhibitory concentration) was determined by testing in a broad concentration range of 0.001, 0.01, 0.1, 1 and 10 μM. (Dennis Wegener et al, Anal. Biochem, 321, 2003, 202-208).

Results for HDAC inhibition at 1 and 10 μM and IC₅₀ values are indicated in table:

TABLE 1

| | | | | | | HDAC Inhibition (μM) | |
|---|---|---|---|---|---|---|---|
| | Cell growth inhibition (GI₅₀ in μM) | | | | | HDAC % | HDAC % |
| Example | NCI | | | | | Inhibition | Inhibition |
| No | H460 | HT-29 | A549 | PC-3 | PA-1 | (1 μM) | (10 μM) |
| 1 | 1 ± 0.2 | 0.9 ± 0.8 | — | 2.4 ± 2.5 | 0.01 | 92 | 94 |
| 2 | 2.6 ± 2.0 | 1.9 ± 1.8 | — | 1.7 ± 1.1 | 0.11 | 98 | 100 |
| 3 | 6.5 ± 4.2 | 5.2 ± 3.5 | — | 3.4 ± 0.4 | — | 84 | 96 |
| 4 | 3.2 ± 1.1 | 2.4 ± 0.5 | — | 4.6 ± 1.6 | — | 88 | 94 |
| 5 | 19 ± 1 | 13.3 ± 2.9 | — | 9.2 ± 9.5 | — | 68 | 88 |
| 6 | >100 | 16.3 | — | >100 | — | 75 | 87 |
| 7 | 3.1 ± 1.7 | 1.4 ± 0.5 | — | 2.6 ± 1.2 | — | 90 | 94 |
| 8 | 2.1 | 0.2 | 4.1 ± 0.6 | 16.8 ± 8.8 | 0.15 ± 0.05 | 95 | 100 |
| 9 | — | — | 5.0 ± 1.6 | 22 ± 5.4 | 0.22 ± 0.03 | 91 | 99 |
| 10 | — | — | 3.2 ± 1.4 | 18.5 ± 10.2 | 0.1 ± 0.02 | 96 | 100 |
| 16 | 0.4 | 0.04 | 1.7 ± 1.0 | 12.6 ± 7.6 | 0.09 ± 0.04 | 99 | 100 |
| 17 | 7.06 ± 6.98 | 0.38 ± 0.12 | — | 4.4 | — | 93 | 99 |
| 18 | 2.21 | >100 | — | — | — | 98 | 100 |
| 19 | 3.38 | 0.2 | — | — | — | 98 | 101 |
| 20 | — | — | 2.9 ± 1.8 | 12.7 ± 5.6 | 0.16 ± 0.02 | 96 | 100 |
| 21 | — | — | 2.2 ± 2.3 | 11.3 ± 8.3 | 0.11 ± 0.04 | 98 | 100 |
| 22 | 0.1 ± 0.2 | 0.5 ± 0.4 | — | 0.6 ± 0.6 | 0.01 | 92 | 96 |
| 23 | 0.3 | 0.2 | — | 12.2 | — | 96 | 99 |
| 24 | 10.65 | 0.48 | — | — | — | 96 | 101 |
| 25 | 0.8 | 0.2 | 2.5 ± 0.8 | 4 ± 3.9 | 0.04 ± 0.01 | 100 | 101 |
| 26 | — | — | 0.9 ± 0.3 | 9.4 ± 6.2 | 0.03 | 99 | 99 |
| 27 | — | — | 0.4 ± 0.2 | 4.5 ± 5.7 | 0.02 | 99 | 99 |
| 28 | — | — | — | — | — | 84 | 96 |
| 29 | — | — | — | — | — | 87 | 98 |
| 30 | — | — | — | — | — | 92 | 99 |
| 31 | — | — | — | — | — | 85 | 97 |
| 32 | — | — | — | — | — | 79 | 91 |
| 33 | — | — | — | — | — | 63 | 92 |
| 34 | 19.9 | 23.8 | — | 23.3 | — | 91 | 99 |
| 35 | 20.4 | 19.3 | — | 47.7 | — | 67 | 92 |

For selected compounds, IC₅₀ (50% HDAC inhibitory concentration) values are given in below table 2:

TABLE 2

| Example No | IC₅₀ (nM) |
|---|---|
| Reference | 78 |
| compound | |
| (SAHA) | |
| 8 | 3 |
| 9 | 57 |
| 10 | 25 |
| 11 | 18.6 |
| 12 | 12.8 |
| 13 | 19.1 |
| 14 | 13.4 |
| 15 | 7.33 |
| 16 | 4 |
| 20 | 19 |
| 21 | 8 |
| 25 | 1 |
| 26 | 1 |
| 27 | 1 |

### Example 38

### Absolute Aqueous Solubility

The objective of the study was to determine the absolute solubility of the powder form of the test compounds in water. Brief procedure: The powder form of test compounds was allowed to saturate in an aqueous medium and is equilibrated for about 6 hrs until the compound precipitates. The precipitated solution was centrifuged at 15,000 rpm for 10 mins at 25 deg centigrade and the supernatant solution was analyzed by UV spectrometry. If required the supernatant was diluted further until the absorbance by UV spectroscopy was within the limits of the standard curve obtained with the test compound. λmax was selected from UV spectra having maximum absorbance for that compound.

TABLE 3
Absolute Aqueous Solubility of HDAC Inhibitors
Absolute Aqueous Solubility of HDAC Inhibitors

| Compound | Solubility (mg/mL) |
|---|---|
| SAHA | 0.23 |
| Ex. 8 | 5.1 |
| Ex. 9 | 4.7 |
| Ex. 16 | 9.4 |
| Ex 21 | 11.6 |
| Ex. 25 | 2.9 |

The results indicated that compounds were 11 to 50 times more soluble as compared to reference compound SAHA

### Example 39

### Metabolic Stability

The objective of the study is to determine the metabolic stability of the compounds in mouse liver microsomes. Brief procedure: metabolic stability of test compounds were carried out using mouse liver microsomes. The final composition of the assay includes test compound 100 μM (dissolved in DMSO), mouse microsomal protein 0.5 mg/mL and cofactors (G-6-P 5.0 mM, G-6-PDH 0.06 U, MgCl2 2.0 mM, NADP+1.0 mM, UDPGA 0.5 mM, PAPS 0.6 mM and GSH 1 mM). The test compounds were incubated at with mouse liver microsomes with cofactors. After 1 h incubation at 37° C., the reaction was stopped by addition of stop solution (ice cold acetonitrile). The samples were centrifuged and supernatants were analysed using LC/MS/MS. The Percent of parent test compounds remaining after 1 h of incubation time was calculated with respect to the peak areas of at time 0.

The results indicated that the test compounds were metabolically more stable as compared to reference compound SAHA.

TABLE 4
Metabolic stability of HDAC inhibitors in mouse liver microsomes
Metabolic stability of HDAC inhibitors in mouse liver microsomes

| | % Stable compounds |
|---|---|
| Compound | after 1 hr incubation |
| SAHA | 67 |
| Ex. 8 | 100 |
| Ex. 9 | 100 |
| Ex. 16 | 98 |
| Ex. 21 | 65 |
| Ex. 25 | 57 |

### Example 40

### hERG Binding Determination

The study was carried out to find our cardio safety of the test compounds. Brief procedure: The hERG competitive binding assay performed at a ligand concentration of 1 nM. The test compounds were dissolved in desired volume of 100% DMSO (stock conc. 50 mM). 10× stocks of the test compounds were made in the assay buffer and 5 μL of stock have been added into wells containing 10 μg hERG-CHO membrane in 35 μL of assay buffer. The test compound and membrane was pre-incubated for 10 Min at 30° C. 10 μL of ³H-Astemizole was then added (final concentration of 1 nM), mixed well and incubated additionally for 90 min at 30° C. with mild shaking. At the end of reaction, binding was terminated by rapid filtration onto GF/C glass fiber filter mats, presoaked in 0.3% polyethyleneimine, followed by rapid washing 10 times with ice-cold wash buffer. Captured radiolabel was detected using a liquid scintillation counter. The percent inhibition of the compounds was calculated compared to the vehicle control.

For the IC₅₀ study of the test compounds, a log concentration of the compound was used in the assay (0.1-300 μM) Astemizole were tested at (0.001-100 μM).

The results in showed that the test compounds have liability towards hERG.

TABLE 5
hERG binding of HDAC inhibitors
hERG binding of HDAC inhibitors (IC₅₀ μM)

| SAHA | Ex. 8 | Ex. 9 | Ex. 16 | Ex. 21 | Ex. 25 |
|---|---|---|---|---|---|
| >100 | 19.91 | >100 | >100 | 42.62 | >100 |

### Example 41

### Cytochrome-P450 Isoforms Liability Determination

To determine the test compound liability towards Human cytochrome 450 (hCYP450) Isoforms fluorescence based screening kits.

Brief procedure: 2× test compounds were prepared by dilution with deionized water. A serial dilution of the test compound was done. 2× solution of known inhibitor were used as a positive control. 40 μL of the 2× solution that is prepared was added to the desired wells. Two replicates were included for every compound. 50 μL of the Master premix (premix of CYP450 Baclosomes, reagents and regenerating system) was dispensed into each well. The plate was incubated for 20 min at room temperature to allow the compounds to interact with the CYP450enzymes. The reaction was started by adding 10 μL of the substrate and NADP⁺ mixture. The plate was incubated for the desired amount of time, and then 10 μL of stop reagent was added to each well to quench the reaction. The fluorescence was measured in the fluorescent plate reader at excitation and emission wavelengths as recommended, depending on the substrate used.

The results indicated that the test compounds do not have liability towards major CYP-450 isoforms. The test compounds were found to be better as compared to SAHA.

TABLE 6
CYP liability of HDAC inhibitors
CYP liability of HDAC inhibitors (IC₅₀ in μM)

| hCYP | | | | | | |
|---|---|---|---|---|---|---|
| isoforms | SAHA | Ex. 8 | Ex. 9 | Ex. 16 | Ex. 21 | Ex. 25 |
| CYP 3A4 | 22.1 | 28.3 | 43.2 | 53.3 | 28.5 | 12.6 |
| CYP 1A2 | 9.7 | >60 | >60 | >60 | >60 | >60 |
| CYP 2C9 | 29.7 | >60 | >60 | >60 | >60 | 51.5 |
| CYP 2C19 | 2.9 | >60 | >60 | >60 | >60 | >60 |
| CYP 2D6 | 3.6 | 26 | >60 | 13.2 | >60 | >60 |

### Example 42

### Oral Bioavailability and Pharmacokinetics Studies

The studies were performed to determine the oral bioavailability and pharmacokinetics of test compounds in male Balb/c mice. Study was performed after obtaining the Institutional Animal Ethics Committee (IAEC) permission. Mice aged 5 to 6 weeks weighing around 25 to 30 g were used for this study. The animals were fasted overnight with free access to water. Animals were administered test compounds by oral route at a 50 mg/kg body weight (formulation: 0.5% methylcellulose in water and 0.1% tween 80. Dose volume 10 ml/kg b.w.) or intravenous route at a dose 10 mg/kg b.w. 50 mg/kg body weight (formulation: 0.9% saline. Dose volume 10 ml/kg b.w.) Blood samples were collected at various time points during the next 24 hours post dose. The blood samples were centrifuged at 3000 g for 5 minutes at 4° C. and the corresponding plasma samples were collected in clean pre-labeled tubes. Then, each sample was subjected to a suitable extraction method and analysed by LC-MS/MS (API 3200 LC-MS/MS system). Data was analyzed using WinNonlin version 5.2 (Pharsight).

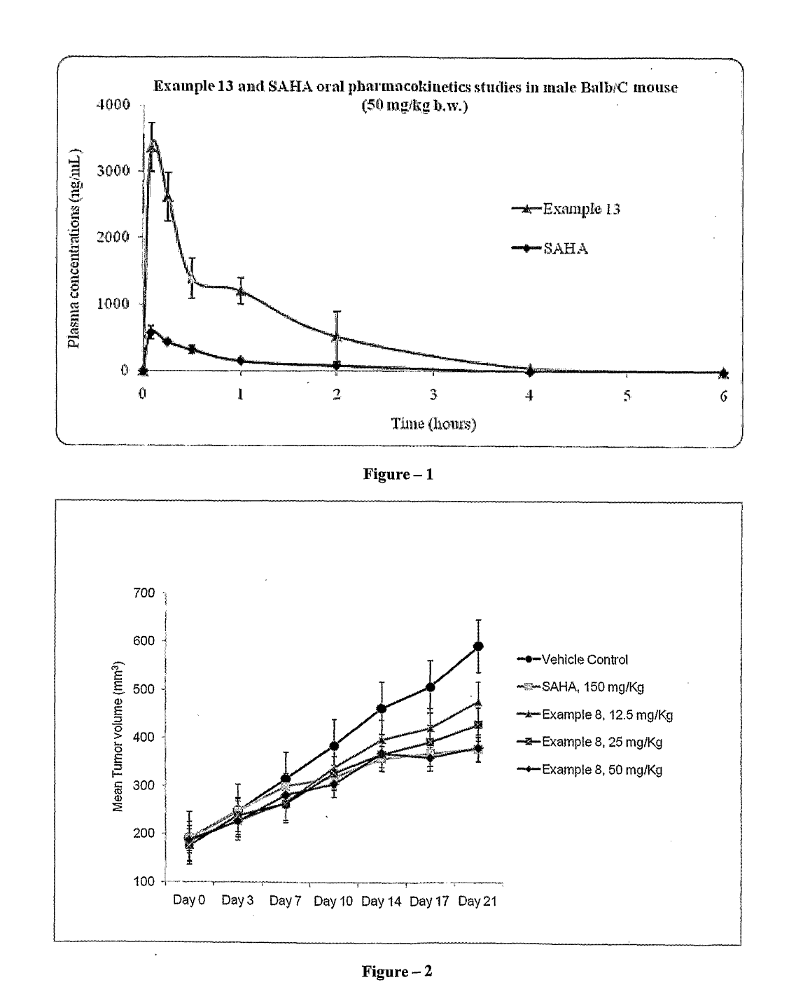

The results indicated that the test compound 11 was 5 times higher Cmax, 2 times longer terminal half life and 9 times higher AUC. FIG. 1 illustrates the same.

TABLE 7
Oral pharmacokinetics parameters of SAHA and
Ex. 13 in male Balb/c mouse
Oral pharmacokinetics parameters of HDAC inhibitors
in male Balb/c mouse

| | Mean Oral pharmacokinetics | |
|---|---|---|
| | parameters | |
| Parameters | SAHA | Ex. 13 |
| Dose (mg/kg) | 50 | 50 |
| Tₘₐₓ (h) | 0.08 | 0.08 |
| Cₘₐₓ (ng/ml) | 580 | 3359 |
| T_{1/2} | 0.8 | 2 |
| AUC last _{0 to 24 h} (h*ng/mL) | 347 | 3340 |
| F% | 11 ± 3 | 68 ± 6 |

### Example 43

### Isoform Selectivity Assay

Following the HDAC inhibition assay using Hela nuclear extract, isoform selectivity was tested using recombinant HDAC isoforms (Biomol, USA). Ex.8 was tested against HDAC1, HDAC2, HDAC3, HDAC6, and HDAC8 isoforms. Results indicate that the compound is a pan-HDAC inhibitor similar to the reference compound SAHA.

TABLE 8

| | IC₅₀ (nM) | | | | | |
|---|---|---|---|---|---|---|
| Example | Hela nuclear | | | | | |
| No | extract | HDAC1 | HDAC2 | HDAC3 | HDAC6 | HDAC8 |
| SAHA | 78 | 83 | 78 | 32 | 128 | 1612 |
| 8 | 3 | 25 | 29 | 2 | 11 | 282 |

TABLE 9
Anti-proliferative activity (GI50) of selected compounds:

| Tissue | Cell line | Ex. 16 | Ex. 8 | Ex. 25 | Ex. 9 | Ex. 21 | SAHA |
|---|---|---|---|---|---|---|---|
| Lung | A549 | 1.58 ± 1.1 | 3.1 ± 1.99 | 2.0 ± 1.52 | 4.5 ± 2.33 | 2.2 ± 2.22 | 5.9 ± 2.4 |
| | NCI-H23 | 0.52 ± 0.04 | 0.9 ± 0.25 | 0.4 ± 0.09 | 2.3 ± 1.14 | 0.5 ± 0.24 | 3.2 ± 1.0 |
| | NCI-H460 | 0.3 ± 0.04 | 1.9 ± 0.75 | 0.45 ± 0.28 | 2.4 ± 0.85 | 1.9 ± 0.74 | 4.4 ± 1.4 |
| | Calu-6 | 0.41 ± 0.16 | 0.6 ± 0.33 | 0.6 ± 0.18 | 1.4 ± 0.31 | 0.4 ± 0.12 | 3.5 ± 0.5 |
| Cervix | Ca Ski | 0.42 ± 0.15 | 1.1 ± 0.49 | 0.4 ± 0.28 | 1.2 ± 0.22 | 1.1 ± 0.73 | 6.0 ± 5.3 |
| | Hela-229 | 1.0 ± 0.27 | 1.1 ± 0.23 | 0.6 ± 0.38 | 1.4 ± 0.26 | 0.7 ± 0.47 | 4.7 ± 3.2 |
| | Hela-S3 | 0.23 ± 0.12 | 0.4 ± 0.09 | 0.2 ± 0.02 | 0.6 ± 0.16 | 0.2 ± 0.08 | 2.9 ± 0.5 |
| Colon | Colo-205 | 0.31 ± 0.06 | 0.3 ± 0.05 | 0.2 ± 0.03 | 0.3 ± 0.071 | 0.4 ± 0.04 | 1.6 ± 0.1 |
| | HCT-15 | 9.8 ± 4.3 | 19.3 ± 3.8 | 1.7 ± 0.89 | 4.1 ± 3.2 | 1.8 ± 1.14 | 5.2 ± 1.9 |
| | HCT-116 | 0.10 ± 0.11 | 0.1 ± 0.04 | 0.2 ± 0.05 | 1.2 ± 0.018 | 0.2 ± 0.04 | 2.2 ± 0.0 |
| | HT-29 | 0.15 ± 0.13 | 1.3 ± 0.33 | 0.4 ± 0.13 | 1.5 ± 0.65 | 2.1 ± 0.46 | 3.6 ± 2.6 |
| Brain | IMR-32 | 0.25 ± 0.07 | 0.4 ± 0.09 | 0.2 ± 0.02 | 0.9 ± 0.201 | 0.2 ± 0.04 | 1.8 ± 0.2 |
| | U-87-MG | 0.76 ± 0.66 | 0.5 ± 0.09 | 1.1 ± 0.73 | 2.7 ± 0.74 | 1.0 ± 0.33 | 6.7 ± 1.1 |
| | SH-SY-5Y | 0.34 ± 0.033 | 0.2 ± 0.08 | 0.2 ± 0.03 | 0.2 ± 0.06 | 0.1 ± 0.0 | 0.8 ± 0.4 |
| Renal | ACHN | 0.09 ± 0.02 | 0.3 ± 0.14 | 0.2 ± 0.08 | 0.7 ± 0.11 | 0.1 ± 0.04 | 1.6 ± 0.5 |
| | 786-O | 2.65 ± 0.08 | 4.1 ± 1.4 | 1.5 ± 0.70 | 2.5 ± 1.01 | 2.0 ± 1.1 | 4.4 ± 2.0 |
| Leukemia | RPMI-8226 | 0.15 ± 0.05 | 0.4 ± 0.3 | 0.2 ± 0.25 | 0.5 ± 0.43 | 0.3 ± 0.16 | 2.2 ± 2.3 |
| | K562 | 0.19 ± 0.05 | 0.3 ± 0.06 | 0.2 ± 0.08 | 0.3 ± 0.074 | 0.2 ± 0.04 | 2.2 ± 0.7 |
| Prostate | DU-145 | 0.079 ± 0.04 | 0.2 ± 0.06 | 0.1 ± 0.03 | 0.3 ± 0.124 | 0.1 ± 0.04 | 1.3 ± 0.0 |
| | PC-3 | 3.5 ± 2.7 | 4.1 ± 0.38 | 1.7 ± 1.02 | 13.4 ± 3.3 | 4.8 ± 1.4 | 8.3 ± 0.6 |
| Pancreas | PANC-1 | 1.03 | 1.3 | 4 | 6.6 | 1.4 | 21.9 |
| Skin | A431 | 0.28 ± 0.05 | 0.5 ± 0.13 | 0.2 ± 0.03 | 1.2 ± 0.533 | 0.4 ± 0.08 | 1.9 ± 1.0 |
| Bone | KHOS | 11.7 ± 1.1 | 2.4 ± 0.4 | 4.6 ± 0.55 | 2.8 ± 0.36 | 10.3 ± 3.26 | 29.3 ± 7.2 |
| Breast | MCF-7 | 3.5 ± 2.6 | 2.6 ± 1.26 | 2.1 ± 2.63 | 5.7 ± 1.62 | 5.5 ± 1.2 | 5.5 ± 1.2 |
| Ovary | SK-OV-3 | 0.04 ± 0.016 | 1.6 ± 0.10 | 0.5 ± 1.2 | 1.8 ± 0.20 | 1.7 ± 0.16 | 4.4 ± 1.5 |
| | PA-1 | 0.08 ± 0.04 | 0.1 ± 0.01 | 0.04 ± 0.01 | 0.2 ± 0.037 | 0.1 ± 0.01 | 0.3 ± 0.05 |

### Example 44

### Effect of Selected Compounds on Histone Hyperacetylation, P21 Induction, Angiogenesis, PARP Cleavage, Cell Differentiation and Caspase-3 Activation

Histone Hyperacetylation in Hela Cells

HeLa Cells were seeded at a density of 0.4×10⁶ Cells/well into 6 well plates and incubated for 24 h The compounds were tested at 5 different concentrations (0.03, 0.1, 0.3, 1.0, 3.0 μM) with corresponding control. After 24 hrs cells were lysed and histone proteins were extracted as per established protocols and the extract was used for measuring H3 and H4 hyperacetylation by Western Blot using Anti-acetyl-Histone H3 and Anti-acetyl-Histone H4 (Millipore, USA) antibodies.

p21 Induction

HeLa Cells were seeded at a density of 0.5×10⁶ Cells/well into 6 well plates and incubated for 24 hrs. Compounds were tested at 5 different concentrations (0.03, 0.1, 0.3, 1.0, 3.0 μM) including corresponding controls. After 24 hrs cells were lysed and the cellular extract was used for measuring P21 induction by Western Blot using monoclonal Anti-p21 clone CP74 antibody (Sigma).

PARP Cleavage

HeLa Cells were seeded (0.3×10⁶ Cells/well) into a 6 well plate and after 24 hrs compounds were added in 5 different concentrations (0.03, 0.1, 0.3, 1.0, 3.0 μM). After 24 hrs cells were lysed and cellular extract was used for assessing apoptotic activity by detection of cleaved PARP by Western Blot using monoclonal Anti-poly(ADP-Ribose) polymerase antibody, Clone C-2-10(Sigma).

Differentiation Assay

HL-60 (AML) cells were seeded (5×10⁴ cells/well) into a 96 well plate and after 24 hrs cells were treated for 3 days with compounds in 8 different concentration (3000, 1000, 300, 100, 30, 10, 3, 1 nM) including control and 60 μM of H2 DCF-DA probe added to the cells. After 2 hours incubation oxidation of H2 DCF-DA was measured.

Angiogenesis Assay

HUVEC cells (7×10⁴ cells/well) cultured in Matrigel into a 24 well plate were treated with different concentrations (12.5, 6.25 and 3.125 μM) of the compounds including positive (Tranilast) and negative control. Incubated overnight (12 to 20 hr) in a 37° C., 5% CO₂ humidified incubator and next day inhibition of the tube formation was monitored under a microscope.

Caspase-3 Activity

Caspase-3 activity was measured in HT-29 cells using Caspase-3 assay kit, (Sigma). HT-29 cells were seeded (10,000 cells/well) in a 96 well plate and incubated overnight. Cells were treated with several concentrations (30, 10, 3, 1, 0.3, 0.1, 0.03, 0.01 μM) of the compounds and incubated for 48 hours and the cells were lysed. Assay was carried out according to the manufacturer's protocol. The fluorescent substrate was added in to the cell lysate and the fluorescence was measured at λₑₓᵢ360 and λₑₘᵢ460.

Results:

The effect of the compounds on secondary assays including histone hyperacetylation, P21 induction, angiogenesis, PARP cleavage, cell differentiation and caspase-3 activation have been tabulated below. The effect on Histone acetylation, P21 induction and PARP cleavage are represented by the symbol ‘+’ which indicates the relative extent of modulation. Angiogenesis is represented by a tick symbol indicating the inhibition of tube formation in the HUVEC angiogenesis assay.

TABLE 10

| Example No. | | SAHA | 16 | 8 | 25 | 9 | 21 |
|---|---|---|---|---|---|---|---|
| Target modulation | H3 acetylation | +++ | ++ | +++ | ++++ | ++++ | +++ |
| | H4 acetylation | +++ | ++++ | ++++ | ++++ | ++++ | ++++ |
| Secondary assays | P21 induction | +++ | + | + | ++ | + | +/++ |
| | Angiogenesis | ✓ | ✓ | ✓ | ✓ | ✓ | ✓ |
| | PARP cleavage | ++ | ++++ | +++ | +++ | +++ | ++++ |
| | Differentiation | 3.06 | 2.88 | 8.24 | 3.831 | ND | ND |
| | EC₅₀ (μM) | | | | | | |
| | Caspase-3 EC₅₀ (μM) | 4.52 | 6.59 | 1.36 | 3.62 | 1.50 | 4.09 |

ND: Not done

### Example 47

### In Vivo Anti-Cancer Activity Using a Human Tumor Xenograft

The study was carried out in athymic nude mice (nu/nu) of BALB-c background at age 6-8 weeks. The animals were maintained in Individually Ventilated cages in a protected and controlled environment. All animal handling and procedures were carried out in a laminar air flow hood, under anesthesia when necessary. The studies were conducted in compliance with protocols approved by Anthem Biosciences Institutional Animal Ethics Committee (IAEC).

Human cell lines derived from tumors of lung were selected for evaluation. The tumor cells cell lines were grown and expanded in RPMI 1640 (supplemented with 1.5 mM L-glutamine and 10% FBS) Subconfluent monolayers were harvested, pelleted and resuspended in RPMI 1640 medium prior to counting on a haemocytometer. Viable cells were counted using trypan blue exclusion and a cell suspension was made in cold media with a concentration of 5×106/ml.

0.3 ml of the cell suspension containing 10⁶ cells of greater than 90% viability was mixed with an equal volume of Matrigel (10 mg/ml) in PBS (pH7.4) and was kept at 4 deg C. Nude mice were anaesthetized and were injected subcutaneously in the flank region with 0.1 ml using a 25 gauge needle. Animals were monitored daily during the period between inoculation and palpable tumor growth. Tumor size was measured with a digital Vernier caliper and tumor-bearing mice were randomized into control and treatment groups (n=10) when sufficient tumor volume was attained (approx. 100 mm3). The following formula was used to calculate the tumor volume:

Tumor volume=(length×width2)/2.

Tumor bearing mice were administered the test compounds at a dose of 12.5, 25 and 50 mg/kg through oral route. Tumor volume and was body weight was measured three times per week.

TABLE 11

| Groups | % TGI on Day 21 |
|---|---|
| Vehicle control | |
| SAHA, 150 mg/kg, p.o | 53.6 |
| Ex. 8, 12.5 mg/kg, p.o | 27.3 |
| Ex. 8, 25 mg/kg, p.o | 36.9 |
| Ex. 8, 50 mg/kg, p.o | 52.1 |

TGI: Tumor growth inhibition with respect to Vehicle treated group

In vivo anti-tumor efficacy was studied in A549 human lung xenograft. After once daily oral administration for 21 days, Ex.8 showed a tumor growth inhibition (TOT) of 27%, 32% and 57% at 12.5, 25 and 50 mg/Kg respectively. The TGI at 50 mg/Kg dose of Ex. 8 was similar to that observed with SAHA at 150 mg/Kg.

The results indicate that Ex. 8 treatment resulted in a significant tumor growth inhibition in a subcutaneous A549 lung tumor xenograft model after 21 days. The efficacy was achieved at 1/3^{rd} the dose of the reference compound SAHA. Furthermore, there was no significant reduction in body weight in the compound treated group compared to the vehicle control group.

We claim:

1. A compound of formula (I), 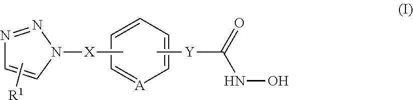
its derivatives, analogs, tautomeric forms, stereoisomers; polymorphs, solvates, salts, metabolites and prodrugs wherein,
R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein;
R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;
X is either absent or is selected from a group comprising cycloalkyl, —(CH₂)ₙ—, —(CH₂)ₙ—NR^{b}—CO—(CH₂)ₙ—, —(CH)ₙR^{a}—NR^{b}—CO—(CH₂)ₙ—, —(CH)ₙR^{a}—NR^{b}—CO—(CH)ₙR^{c}—, —(CH₂)ₙ—NR^{b}—CO—(CH)ₙR^{c}—, —(CH₂)ₙ—NR^{b}—CO—(CH₂)ₙ—, —(CH)ₙR^{a}—NR^{b}—SO ₂—(CH₂)ₙR^{c}—, —(CH)ₙR^{a}—NR^{b}—SO₂—(CH)ₙ—R^{c}— and —(CH₂)ₙ—NR^{b}—SO₂—(CH)ₙR^{c}—;
n is an integer selected from 0 to 6;
R^{a} and R^{c} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl;
R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, —C(═O)R^{a}, —C(═O)OR^{a}, —C(═O)NR^{a}R^{c} and —SO₂R^{a};
Y is either absent or selected from a group comprising —CH₂—, —CH₂CH₂—, —CH═CH—, C₃-C₆ cycloalkyl each of which is optionally substituted with a substituent selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl; and
A is selected from a group comprising Carbon and Nitrogen.

2. The compound as claimed in claim 1, having general formula (II), 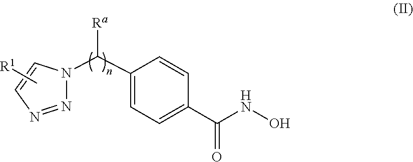
its derivatives, analogs, tautomeric forms, stereoisomers, polymorphs, solvates, salts, metabolites and prodrugs wherein,
R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein ;
R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;
n is an integer equal to 1; and
R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl.

3. The compound as claimed in claim 1, having general formula (III) 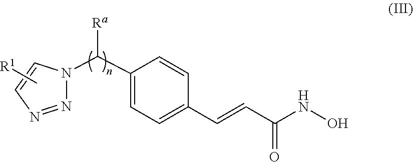
its derivatives, analogs, tautomeric forms, stereoisomers, polymorphs, solvates, salts, metabolites and prodrugs wherein,
R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein ;
R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;
n is an integer equal to 1; and
R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl.

4. The compound as claimed in claim 1, having general formula (IV) 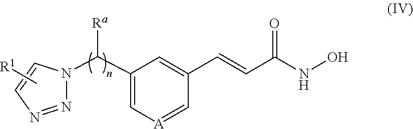
its derivatives, analogs, tautomeric forms, stereoisomers, polymorphs, solvates, salts, metabolites and prodrugs wherein,
R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein;
R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylamino carbonyl, arylaminocarbonyl and heteroarylcarbonyl;
n is an integer equal to 1;
R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl; and
A is selected from a group comprising carbon and nitrogen.

5. The compound as claimed in claim 1, having general formula (V) 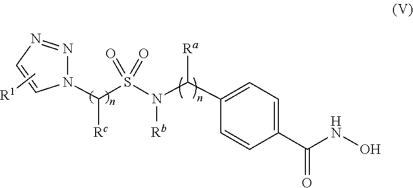
its derivatives, analogs, tautomeric forms, stereoisomers, polymorphs, solvates, salts, metabolites and prodrugs wherein,
R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which is optionally substituted with one or more substituents represented as R² wherein ;
R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;
n is an integer equal to 1;
R^{a} and R^{c} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl; and
R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, —C(═O)R^{a}, —C(═O)OR^{a}, —C(═O)NR^{a}R^{c} and —SO₂R^{a}.

6. The compound as claimed in claim 1, having general formula (VI) 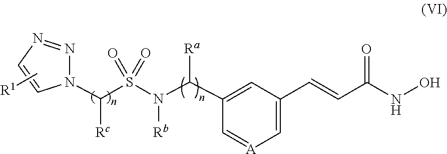
its derivatives, analogs, tautomeric forms, stereoisomers, polymorphs, solvates, salts, metabolites and prodrugs wherein,
R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl each of which may be optionally substituted with one or more substituents represented as R² wherein ;
R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkyl amino carbonyl, arylaminocarbonyl and heteroarylcarbonyl;
n is an integer equal to 1;
R^{a} and R^{c} are independently selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl;
R^{b} is selected from a group comprising hydrogen, alkyl, cycloalkyl, cycloalkylalkyl, hydroxyalkyl, aminoalkyl, heterocyclyl, aryl, araylkyl, hereroaryl, heteroarylalkyl, —C(═O)R^{a}, —C(═O)NR^{a}R^{c} and —SO₂R^{a}; and
A is selected from a group comprising Carbon and Nitrogen.

7. A process for the preparation of compound of formula II, 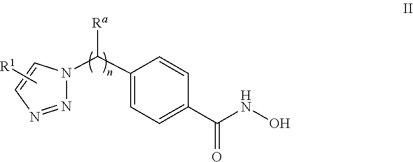
wherein,
R¹ is selected from a group comprising hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl; acylamino, arylamino, COOH, alkoxycarbonyl, alkylaminocarbonyl, arylaminocarbonyl, heteroarylcarbonyl, aryl and heteroaryl each of which may be optionally substituted with one or more substituents selected from R² ;
R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl;
n is an integer equal to 1; and
R^{a} is selected from a group comprising alkyl, cycloalkyl, heterocyclyl, aryl, aralkyl, heteroaryl, heteroarylalkyl, hydroxy, alkoxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl and heteroarylcarbonyl;
said process comprising acts of;
a) converting 1-Bromo-2-fluoro-4-methyl-benzene to an amine;
b) coupling the amine with 4-Azidomethyl-benzoic acid methyl ester in the presence of copper iodide to obtain a triazole compound; and
c) reacting the triazole compound with hydroxylamine in presence of a base to obtain compound of formula II.

8. The process as claimed in claim 7, wherein the base selected from group comprising, sodium methoxide, sodium ethoxide and n-butyllithium, preferably sodium methoxide.

9. A pharmaceutical composition, comprising a compound of formula (I) along with pharmaceutically acceptable excipients(s) selected from a group comprising binders, disintegrants, diluents, lubricants, plasticizers, permeation enhancers and solubilizers.

10. The pharmaceutical composition as claimed in claim 9, wherein the compound of formula (I) is selected from a group comprising compounds of formula (II), formula (III), formula (IV), formula (V), and formula (VI).

11. The pharmaceutical composition as claimed in claim 9, wherein the said composition is in form selected from a group comprising tablet, capsule, powder, syrup, solution, aerosol and suspension.

12. A method of inhibiting Histone deacetylase (HDAC), said method comprising contacting HDAC with a compound of formula (I), or prodrug of compound of formula (I) or pharmaceutical composition comprising compound of formula (I) optionally along with pharmaceutically acceptable excipients.

13. A method of treating disease by HDAC inhibition, said method comprising administering biologically suitable amounts of compound of formula (I), prodrug of compound of formula(I) pharmaceutical composition comprising formula (I) optionally along with pharmaceutically acceptable excipients(s) to a subject in need thereof.

14. The method as claimed in claim 13 wherein the compound of formula (I) is selected from a group comprising compounds of formula(II), formula (III), formula (IV), formula (V), and formula (VI).

15. The method as claimed in claim 13, wherein the subject is an animal, including human beings.
